(19) United States
(12) Patent Application Publication
(10) Pub. No.: US 2024/0352087 A1
(43) Pub. Date: Oct. 24, 2024

(54) T Cell Receptors (TCR) to Human Papillomavirus Proteins, Compositions, and Uses Thereof

(71) Applicant: Emory University, Atlanta, GA (US)

(72) Inventors: Rafi Ahmed, Atlanta, GA (US); Andreas Wieland, Atlanta, GA (US); Christiane Eberhardt, Atlanta, GA (US)

(21) Appl. No.: 18/579,142

(22) PCT Filed: Jul. 14, 2022
(86) PCT No.: PCT/US2022/073733

§ 371 Date: Jan. 12, 2024

Related U.S. Application Data
(60) Provisional application No. 63/221,486, filed on Jul. 14, 2021.

(51) Int. Cl. C07K 14/725

(52) U.S. Cl. CPC C07K 14/7051; C07K 2319/60

(57) ABSTRACT

This disclosure relates T cell receptors (TCR) having antigenic specificity for an epitopes of human papillomavirus (HPV) proteins such as E2, E5, and E6. Related poly peptides and proteins, as well as related nucleic acids, recombinant expression vectors, host cells, and populations of cells are also provided. Pharmaceutical compositions relating to the TCRs are also provided. Also disclosed are methods of detecting the presence of a condition in a mammal and methods of treating or preventing a condition in a mammal, wherein the condition is an epithelial cancer. HPV infection, or HPV-positive premalignancy.

### CROSS-REFERENCE TO RELATED APPLICATIONS

This application claims the benefit of U.S. Provisional Application No. 63/221,486 filed Jul. 14, 2021. The entirety of this application is hereby incorporated by reference for all purposes.

### STATEMENT REGARDING FEDERALLY SPONSORED RESEARCH OR DEVELOPMENT

This invention was made with government support under AI057266 awarded by the National Institutes of Health. The government has certain rights in the invention.

### INCORPORATION-BY-REFERENCE OF MATERIAL SUBMITTED AS AN XML FILE VIA THE OFFICE ELECTRONIC FILING SYSTEM

The Sequence Listing associated with this application is provided in XML format and is hereby incorporated by reference into the specification. The name of the XML file containing the Sequence Listing is 20070PCT.xml. The XML file is 248 KB, was created on Jul. 13, 2022, and is being submitted electronically via the USPTO patent electronic filing system.

### BACKGROUND

The primary cause of some cancer types in epithelial tissues is associated with human papillomavirus (HPV) infections. Treatment of these cancers typically involves surgery, radiation, and chemotherapy agents, which are not always universally effective. Accordingly, there exists a need for additional treatment options.

Draper et al., report targeting of HPV-16+epithelial cancer cells by TCR gene engineered T cells directed against E6. Clin Cancer Res. 2015, 21 (19): 4431-4439.

Krishna et al. report human papilloma virus specific immunogenicity and dysfunction of CD8 T cells in head and neck cancer. Cancer Res, 2018, 78 (21): 6159-6170.

Nagarsheth et al. report TCR-engineered T cells targeting E7 for patients with metastatic HPV-associated epithelial cancers. Nat Med, 2021, 27 (3): 419-425.

See also U.S. Patent Publication No. 2016/0152681.

References cited herein are not an admission of prior art.

### SUMMARY

This disclosure relates T cell receptors (TCR) having antigenic specificity for an epitopes of human papillomavirus (HPV) proteins such as E2, E5, and E6. Related polypeptides and proteins, as well as related nucleic acids, recombinant expression vectors, host cells, and populations of cells are also provided. Pharmaceutical compositions relating to the TCRs are also provided. Also disclosed are methods of detecting the presence of a condition in a mammal and methods of treating or preventing a condition in a mammal, wherein the condition is an epithelial cancer, HPV infection, or HPV-positive premalignancy.

In certain embodiments, the epitopes of human papillomavirus (HPV) proteins are E2, E5, and E6 segments that specifically bind peptides disclosed herein. In certain embodiments, the epitope is a segment comprising or consisting of amino acids 151-159 of E2, QVDYYGLYY (SEQ ID NO: 1). In certain embodiments, the epitope is a segment comprising or consisting of amino acids 329-337 of E2, KSAIVTLTY (SEQ ID NO: 2). In certain embodiments, the epitope is a segment comprising or consisting of amino acids 46-54 of E5, VLLLWITAA (SEQ ID NO: 3). In certain embodiments, the epitope is a segment comprising or consisting of amino acids 55-63 of E5, SAFRCFIVY (SEQ ID NO: 4). In certain embodiments, the epitope is a segment comprising or consisting of amino acids 65-73 E5 of IFVYIPLFL (SEQ ID NO: 5). In certain embodiments, the epitope is a segment comprising or consisting of amino acids 52-61 of E6 FAFRDLCIVY (SEQ ID NO: 6). In certain embodiments, the epitope is a segment comprising or consisting of amino acids 81-90 of E6 SEYRHYCYSL (SEQ ID NO: 7). In certain embodiments, the epitope is a segment comprising or consisting of amino acids 83-90 of E6, YRHYCYSL (SEQ ID NO: 8). In certain embodiments, the epitope is a segment comprising or consisting of amino acids 137-144 of E6 GRWTGRCM (SEQ ID NO: 9). In certain embodiments, the segment is present in complex with or conjugated to a human leukocyte antigen.

In certain embodiments, this disclosure relates to peptide epitopes disclosed herein, derivatives, and conjugated forms or a multimer, e.g., dimer, trimer, or tetramer. In certain embodiments, the multimer comprises a label, e.g., conjugated to a label.

In certain embodiments, this disclosure relates to a cell comprising a T cell receptor (TCR) having antigenic specificity for human papillomavirus (HPV) protein E2, E5, or E6 and comprising a human variable region and a constant region, e.g., heterologous constant region. In certain embodiments, the TCR is isolated or purified and has antigenic specificity for HPV epitope disclosed herein, e.g., the amino acid sequences of SEQ ID NOs: 1-9.

In certain embodiments, this disclosure relates to a T cell receptor (TCR) having specificity for human papillomavirus (HPV) E2, E5, or E6 and comprising a human variable region and a constant region. In certain embodiments, the TCR alpha chain and beta chain comprise the amino acid sequences as disclosed herein or variants thereof.

In certain embodiments, this disclosure relates to an isolated, purified, or chimeric polypeptide comprising a functional portion of a TCR disclosed herein. In certain embodiments, the functional portion comprises the alpha chain CDR1, the alpha chain CDR2, the alpha chain CDR3, the beta chain CDR1, the beta chain CDR2, and the beta chain CDR3.

In certain embodiments, the alpha chain complementarity determining regions CDR1 CDR2, or CDR3 comprise one or two amino acid substitutions, additions, or deletions and TCR retains specific binding to the epitope. In certain embodiments, the beta chain complementarity determining regions CDR1 CDR2, or CDR3 comprise one or two amino acid substitutions, additions, or deletions and TCR retains specific binding to the epitope.

In certain embodiments, this disclosure relates to an isolated, purified, or chimeric protein comprising a polypeptide disclosed herein.

In certain embodiments, this disclosure relates to an isolated, purified, or chimeric protein comprising a first polypeptide chain comprising the alpha chain CDR3 and a second polypeptide chain comprising the beta chain CDR3.

In certain embodiments, this disclosure relates to an isolated, purified, or chimeric protein comprising a first polypeptide chain comprising the alpha chain CDR1, the alpha chain CDR2, the alpha chain CDR3 and a second polypeptide chain comprising the beta chain CDR1, the beta chain CDR2, and the beta chain CDR3. In certain embodiments, the protein is a fusion protein. In certain embodiments, the protein is a recombinant antibody.

In certain embodiments, it is contemplated that TCRs disclosed herein specifically bind HPV-16 proteins, e.g., E2, E5, and E6, through recognition of the epitope complexed with HLAs, human leukocyte antigens. In certain embodiments, this disclosure contemplates methods of isolating human T cells; contacting the T cells with a nucleic acid encoding a TCR disclosed herein thereby expressing the TCRs providing e.g., E2 TCR-T cells, E5 TCR-T cells, and/or E6 TCR-T cells, which are capable of engaging and killing HPV infected tumor cell lines in vitro and mediate regression of HPV infected tumors in vivo.

In certain embodiments, this disclosure relates to a method of treating or preventing cancer in a mammal, comprising administering a population of cells to the mammal in an amount effective to treat or prevent the cancer in the mammal, wherein: the population of cells comprises at least one host cell expressing a T-cell receptor (TCR), or heterologous protein, or variants, having antigenic specificity for HPV 16 for epitopes of E2, E5, or E6 as disclosed herein. In certain embodiments, the cell therapy is in combination with another anticancer agent.

In certain embodiments, this disclosure relates to a method of treating or preventing a condition in a mammal, comprising administering a population of cells to the mammal in an amount effective to treat or prevent the condition in the mammal, wherein: the population of cells comprises at least one host cell expressing a T-cell receptor (TCR) having antigenic specificity for HPV 16 as disclosed herein wherein the population of cells is allogeneic or autologous to the mammal; and the condition is cancer, HPV 16 infection, or HPV-positive premalignancy.

In certain embodiments, this disclosure relates to an isolated or purified nucleic acid comprising a nucleotide sequence encoding the TCR or protein disclosed herein in operable combination with a heterologous promoter. In certain embodiments, this disclosure relates to a recombinant expression vector comprising the nucleic acid encoding a TCR or protein disclosed herein. In certain embodiments, this disclosure relates to an isolated host cell comprising the recombinant expression vector. In certain embodiments, the host cell according is a human cell. In certain embodiments, this disclosure relates to population of cells comprising at least one host cell.

In certain embodiments, this disclosure relates to a method of treating or preventing a condition in a mammal, the method comprising administering to the mammal a population of host cells comprising a recombinant expression vector comprising a nucleic acid comprising a nucleotide sequence encoding a T cell receptor (TCR) having antigenic specificity for human papillomavirus (HPV) 16 E2, E5, or E6 as disclosed herein wherein the condition is cancer, HPV 16 infection, or HPV-positive premalignancy.

In certain embodiments, this disclosure relates to a method of detecting the presence of a condition in a mammal, comprising: (a) contacting a sample comprising one or more cells from the mammal with a TCR as disclosed herein, thereby forming a complex, and (b) detecting the complex, wherein detection of the complex is indicative of the presence of the condition in the mammal, wherein the condition is cancer, HPV 16 infection, or HPV-positive premalignancy. In certain embodiments, the condition is cancer of the uterine cervix, oropharynx, anus, anal canal, anorectum, vagina, vulva, or penis. In certain embodiments, the condition is an HPV 16-positive cancer.

In certain embodiments, this disclosure relates to a pharmaceutical composition comprising a TCR, polypeptide, nucleic acid, or TCR population of cells or other TCR materials as disclosed herein, and a pharmaceutically acceptable carrier.

In certain embodiments, this disclosure relates to using TCRs sequences disclosed herein as targeting agents on particles, nanoparticles, micro sized particles, vesicles, cells, for delivery of an anticancer agent or other payload to HPV 16-positive cancer.

In certain embodiments, this disclosure relates to compositions comprising a polypeptide comprising or consisting of epitopes selected from of SEQ ID NOs: 1-9. In certain embodiments, the polypeptide consists of an N-terminus having the amino acid sequence of SEQ ID NO: 1-9. In certain embodiments, the polypeptide consists of a C-terminus having the amino acid sequence of SEQ ID NO: 1-9. In certain embodiments, the polypeptide is conjugated to a label.

In certain embodiments, this disclosure relates to multimeric complex comprising a polypeptide disclosed herein. In certain embodiments, the polypeptide is specifically binding an MHC-I protein.

In certain embodiments, this disclosure relates to methods comprising: contacting a polypeptide or multimeric complex of a polypeptide of SEQ ID NOs: 1-9 with a sample from a subject at risk of, exhibiting symptoms of, or diagnosed with cancer comprising T cells with receptors that specifically bind the polypeptide providing polypeptide bound T cells; and isolating the polypeptide bound T cells providing isolated T cells that bind to the polypeptide.

In certain embodiments, the sample comprises tumor-infiltrating lymphocytes isolated from primary tumors or metastatic lymph nodes from the subject. In certain embodiments, the isolated T cells are CD8+.

### BRIEF DESCRIPTION OF THE SEVERAL VIEWS OF THE DRAWINGS

FIG. 1A shows a schematic of the experimental strategy to map HPV-specific CD8 T cell epitopes in HNSCC patients. Expanded PBMCs of an HLA-A*01: 01+ individual stimulated with a peptide, e.g., HPV E2329-337. and analyzed by Interferon-y ELISPOT, ICCS, or stained with the respective tetramer. Flow plots were gated on CD3+CD4-CD8+ T cells.

FIG. 1B shows a table summarizing results of the HLA-binding assays (IC₅₀) of identified HPV epitopes. In bold are indicated epitopes for which ex vivo tetramer staining was performed.

FIG. 1C show a graph summarizing the frequency of tetramer-positive cells in primary tumors, metastatic LN and peripheral blood. Tetramers are coded and individual patients are represented as distinct shapes. Individual tetramers from matched patient samples are connected with a line.

FIG. 2A shows the sequences of the alpha and beta chains of TCRs identified to bind the specific epitopes.

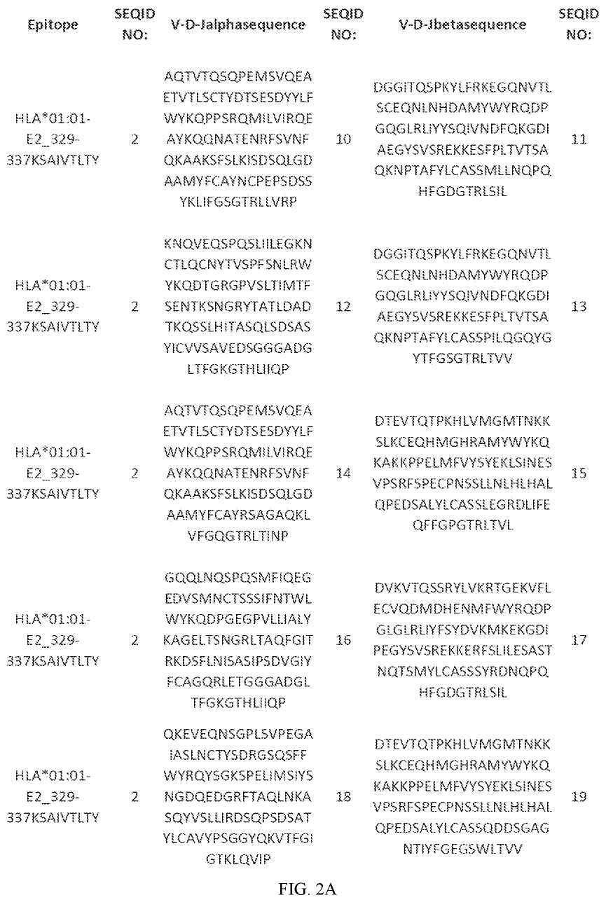

FIG. 2B shows the sequences of the alpha and beta chains of TCRs identified to bind the specific epitopes.

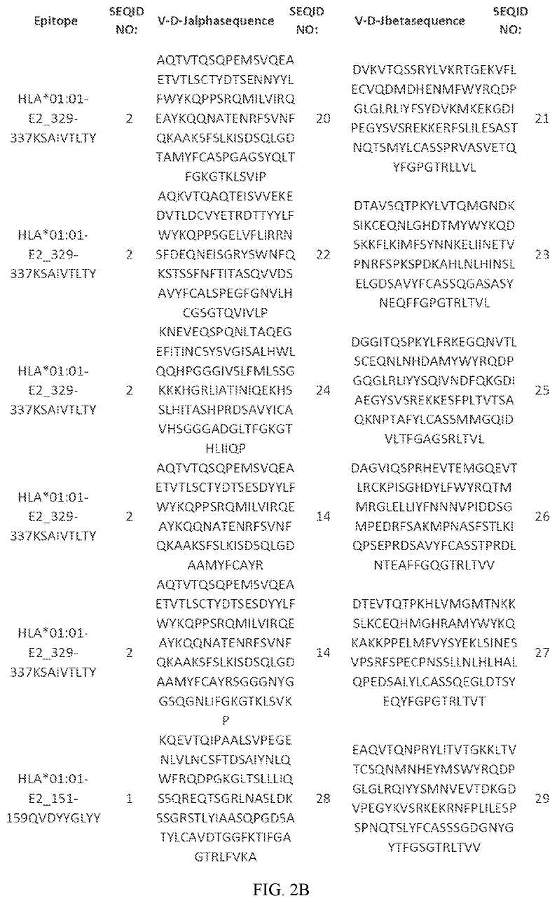

FIG. 2C shows the sequences of the alpha and beta chains of TCRs identified to bind the specific epitopes.

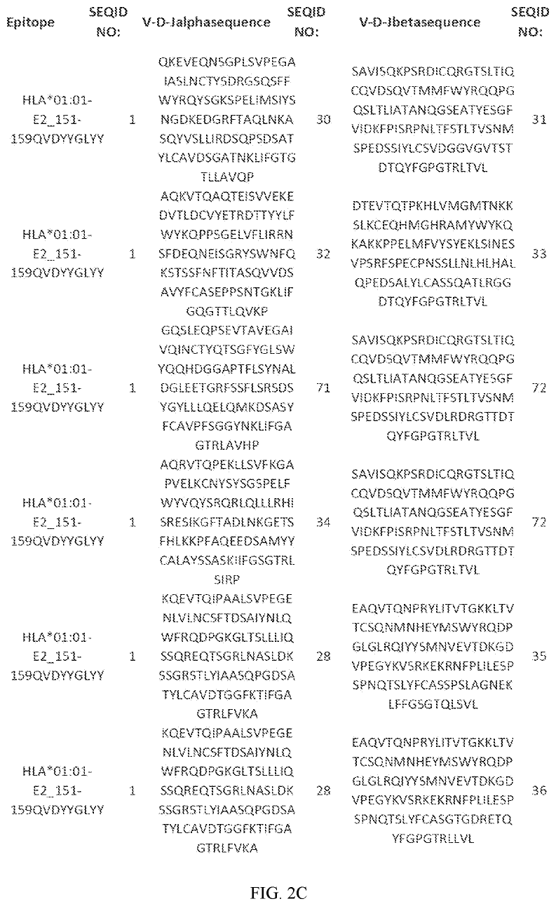

FIG. 2D shows the sequences of the alpha and beta chains of TCRs identified to bind the specific epitopes.

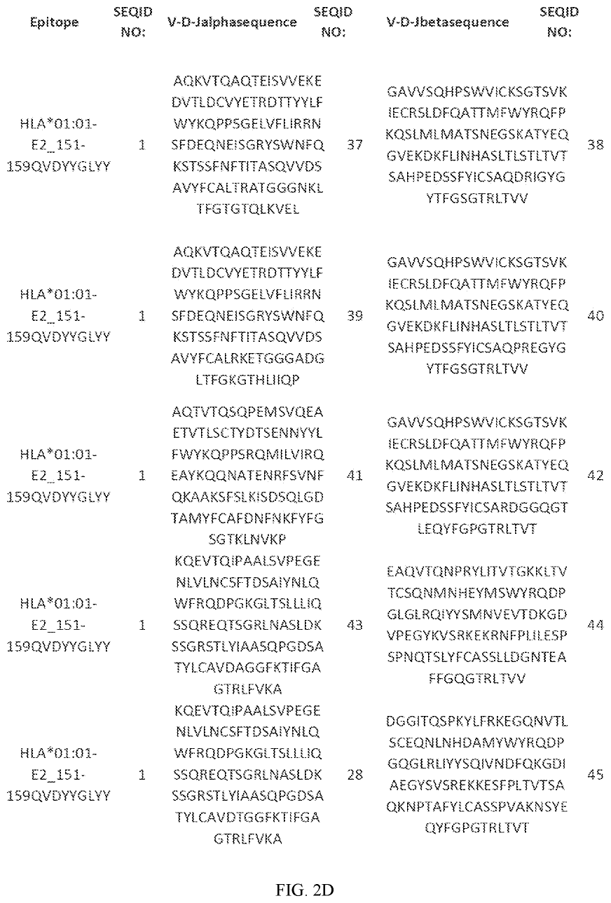

FIG. 2E shows the sequences of the alpha and beta chains of TCRs identified to bind the specific epitopes.

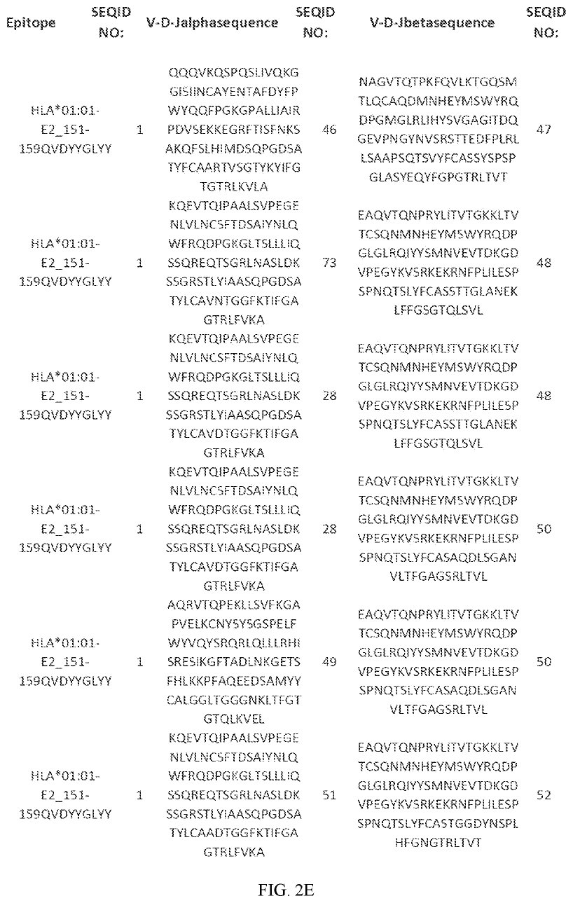

FIG. 2F shows the sequences of the alpha and beta chains of TCRs identified to bind the specific epitopes.

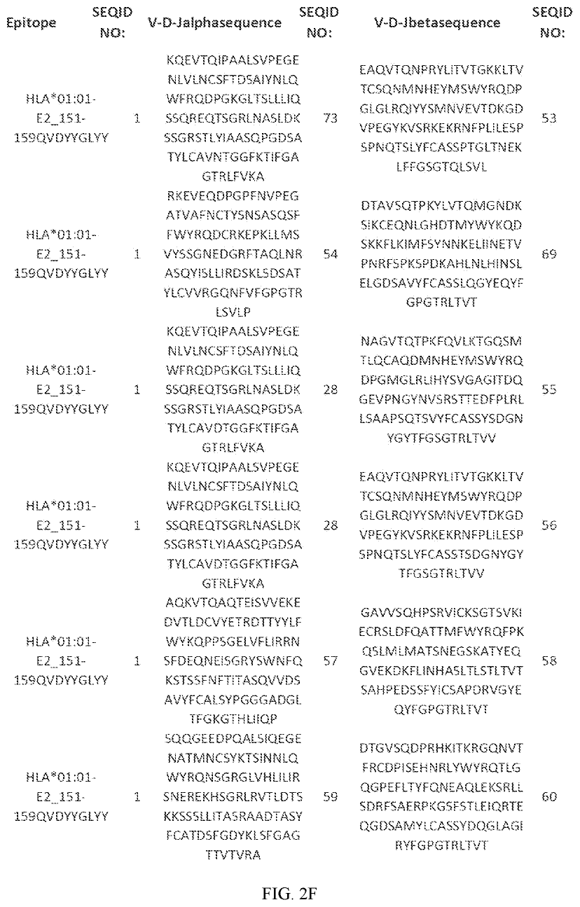

FIG. 2G shows the sequences of the alpha and beta chains of TCRs identified to bind the specific epitopes.

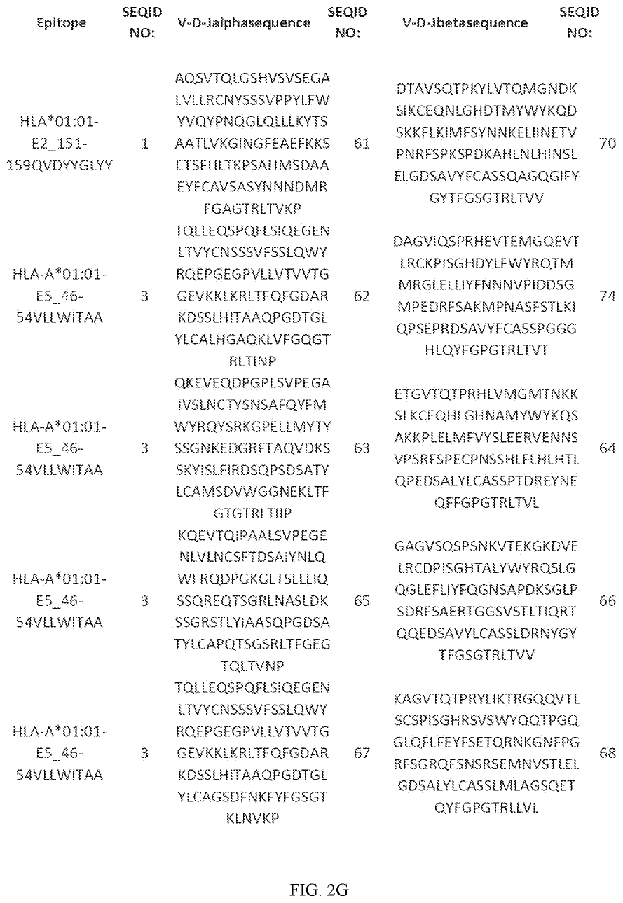

FIG. 3A shows the sequences of the alpha and beta chain CDRs of TCRs identified to bind the specific epitopes.

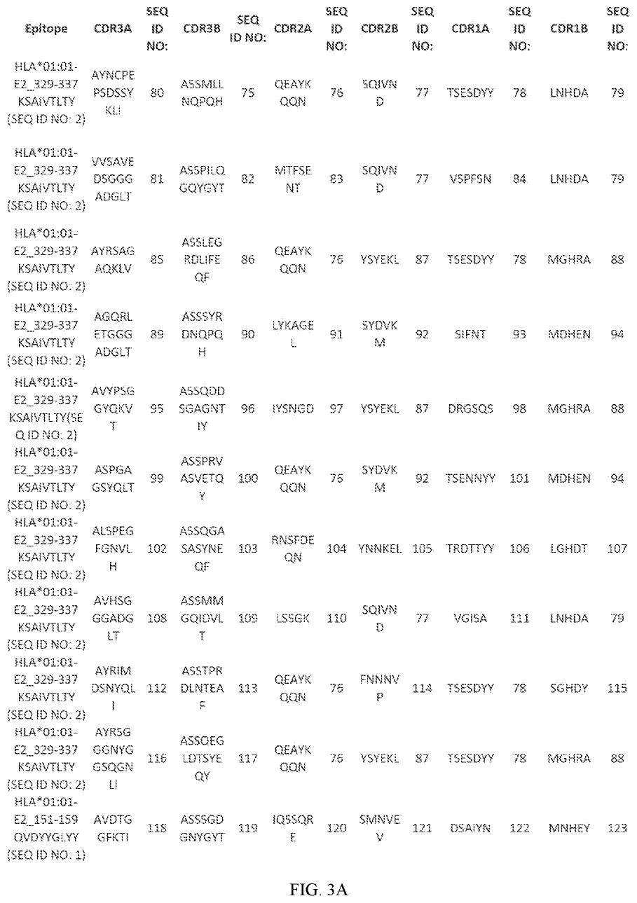

FIG. 3B shows the sequences of the alpha and beta chain CDRs of TCRs identified to bind the specific epitopes.

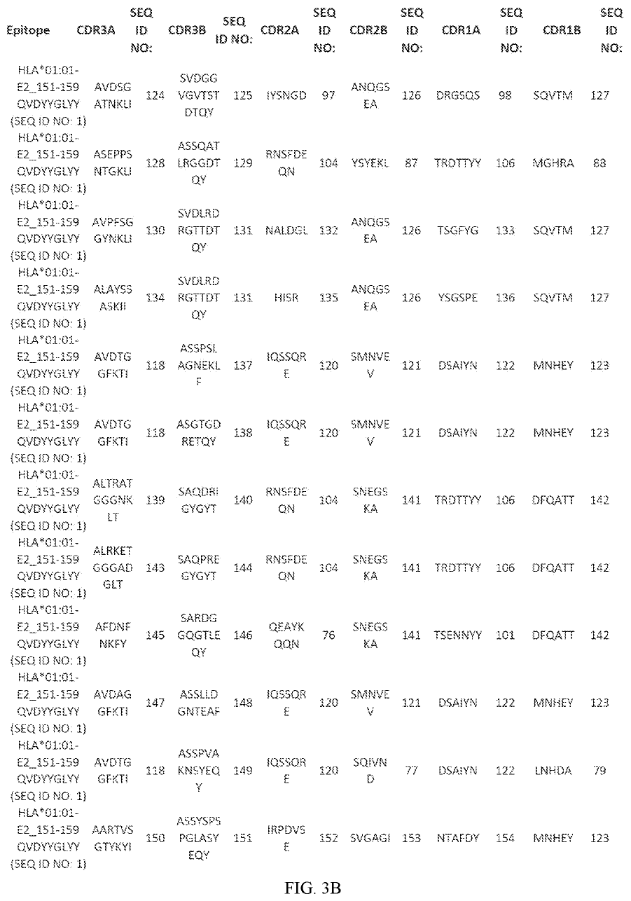

FIG. 3C shows the sequences of the alpha and beta chain CDRs of TCRs identified to bind the specific epitopes.

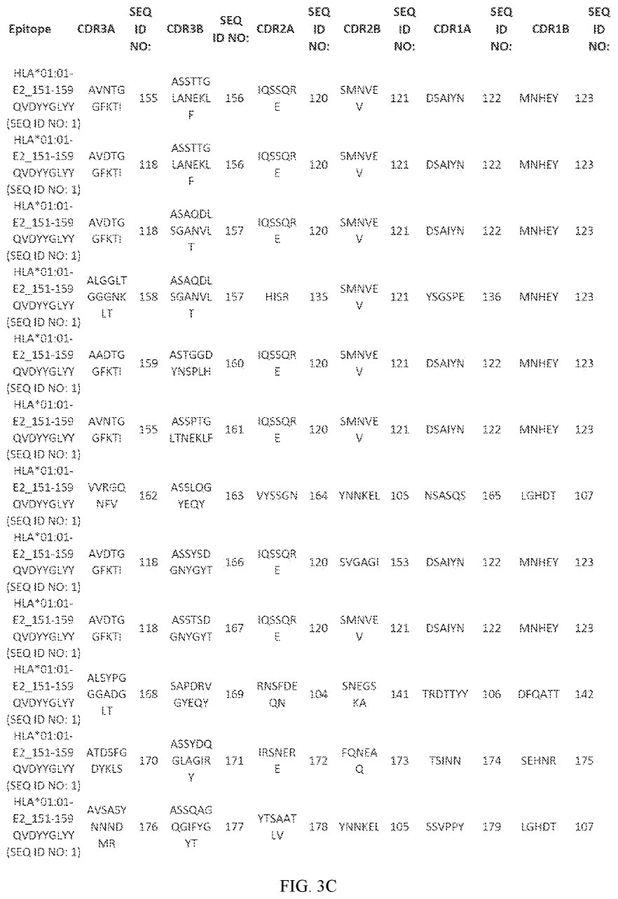

FIG. 3D shows the sequences of the alpha and beta chain CDRs of TCRs identified to bind the specific epitopes.

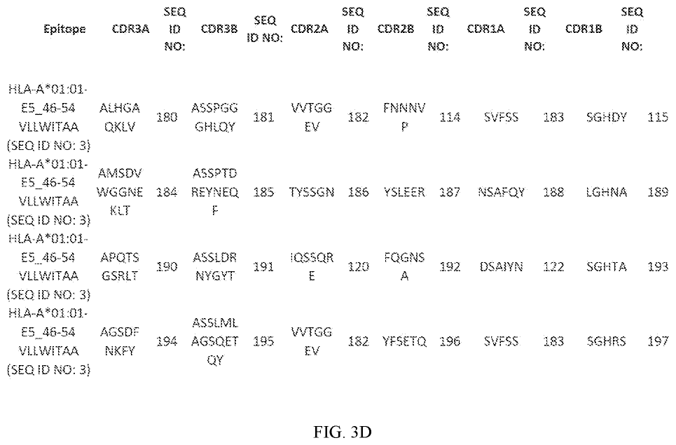

### DETAILED DISCUSSION

Before the present disclosure is described in greater detail, it is to be understood that this disclosure is not limited to embodiments described, and as such may, of course, vary. It is also to be understood that the terminology used herein is for describing particular embodiments only, and is not intended to be limiting, since the scope of the present disclosure will be limited only by the appended claims.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

An “embodiment” of the disclosure refers to an example but not necessarily limited to such an example. Embodiments of the present disclosure will employ, unless otherwise indicated, techniques of medicine, organic chemistry, biochemistry, molecular biology, pharmacology, and the like, which are within the skill of the art. Such techniques are explained fully in the literature.

It must be noted that, as used in the specification and the appended claims, the singular forms “a,” “an,” and “the” include plural referents unless the context clearly dictates otherwise. As such, the terms “a”, “an”, “one or more” and “at least one” can be used interchangeably. Similarly, the terms “comprising”, “including” and “having” can be used interchangeably. It is further noted that the claims may be drafted to exclude any optional element.

The terms “protein,” “peptide,” and “polypeptide” are used interchangeably to refer to compounds comprising amino acids joined via peptide bonds and are used interchangeably. Amino acids may be naturally or non-naturally occurring. A “chimeric protein” or “fusion protein” is a molecule in which different portions of the protein are derived from different origins such that the entire molecule is not naturally occurring. A chimeric protein may contain amino acid sequences from the same species of different species as long as they are not arranged together in the same way that they exist in a natural state. Examples of a chimeric protein include sequences disclosed herein that are contain one, two or more amino acids attached to the C-terminal or N-terminal end that are not identical to any naturally occurring protein, such as in the case of adding an amino acid containing an amine side chain group, e.g., lysine, an amino acid containing a carboxylic acid side chain group such as aspartic acid or glutamic acid, a polyhistidine tag, e.g. typically four or more histidine amino acids. Contemplated chimeric proteins include those with self-cleaving peptides such as P2A-GSG. See Wang. Scientific Reports 5, Article number: 16273 (2015).

A “heterologous” nucleic acid sequence or peptide sequence refers to a nucleic acid sequence or peptide sequence that do not naturally occur, e.g., because the whole sequences contain a segment from other plants, bacteria, viruses, other organisms, or joinder of two sequences that occur the same organism but are joined together in a manner that does not naturally occur in the same organism or any natural state.

The term “recombinant” when made in reference to a nucleic acid molecule refers to a nucleic acid molecule which is comprised of segments of nucleic acid joined together by means of molecular biological techniques provided that the entire nucleic acid sequence does not occurring in nature, i.e., there is at least one mutation in the overall sequence such that the entire sequence is not naturally occurring even though separately segments may occur in nature. The segments may be joined in an altered arrangement such that the entire nucleic acid sequence from start to finish does not naturally occur. The term “recombinant” when made in reference to a protein or a polypeptide refers to a protein molecule that is expressed using a recombinant nucleic acid molecule.

As used herein, the term “derivative” refers to a structurally similar peptide that retains sufficient functional attributes of the identified analogue. The derivative may be structurally similar because it is lacking one or more atoms, e.g., replacing an amino group, hydroxyl, or thiol group with a hydrogen, a salt, in different hydration/oxidation states, or because one or more atoms within the molecule are switched, such as, but not limited to, replacing an oxygen atom with a sulfur atom or a sulfur with an oxygen or replacing an amino group with a hydroxyl group or a hydroxyl with an amino group. The derivative may be a prodrug, comprise a lipid, polyethylene glycol, saccharide, polysaccharide. A derivative may be two or more peptides linked together by a linking group. It is contemplated that the linking group may be biodegradable. Derivatives may be prepare by any variety of synthetic methods or appropriate adaptations presented in synthetic or organic chemistry text books, such as those provide in March's Advanced Organic Chemistry: Reactions, Mechanisms, and Structure, Wiley, 6th Edition (2007) Michael B. Smith or Domino Reactions in Organic Synthesis, Wiley (2006) Lutz F. Tietze hereby incorporated by reference.

In certain embodiments, the peptides disclosed herein have at least one non-naturally occurring molecular modification, such as the attachment of polyethylene glycol, the attachment of a chimeric peptide, the attachment of a fluorescent dye comprising aromatic groups, fluorescent peptide, a chelating agent, a radionuclide such as ¹⁸F, N-terminal acetyl, propionyl group, myristoyl and palmitoyl, group or N-terminal methylation, or a C-terminal alkyl ester. In certain embodiments, the disclosure contemplates the disclosure contemplates peptides disclosed herein labeled using commercially available biotinylation reagents. Biotinylated peptide can be used in streptavidin affinity binding, purification, and detection. In certain embodiments, the disclosure contemplates peptide disclose herein containing azide-derivatives of naturally occurring monosaccharides such as N-azidoacetylglucosamine, N-azidoacetylmannosamine, and N-azidoacetylgalactosamine.

In certain embodiments, this disclosure contemplates derivatives of peptide disclose herein wherein one or more amino acids are substituted with chemical groups to improve pharmacokinetic properties such as solubility and serum half-life, optionally connected through a linker. In certain embodiments, such a derivative may be a prodrug wherein the substituent or linker is biodegradable, or the substituent or linker is not biodegradable. In certain embodiments, contemplated substituents include a saccharide, polysaccharide, acetyl, fatty acid, lipid, and/or polyethylene glycol. The substituent may be covalently bonded through the formation of amide bonds on the C-terminus or N-terminus of the peptide optionally connected through a linker. In certain embodiments, it is contemplated that the substituent may be covalently bonded through an amino acid within the peptide, e.g., through an amine side chain group such as lysine or an amino acid containing a carboxylic acid side chain group such as aspartic acid or glutamic acid, within the peptide comprising a sequence disclosed herein. In certain embodiments, it is contemplated that the substituent may be covalently bonded through a cysteine in a sequence disclosed herein optionally connected through a linker. In certain embodiments, a substituent is connected through a linker that forms a disulfide with a cysteine amino acid side group.

As used herein, the term “conjugated” refers to linking molecular entities through covalent bonds, or by other specific binding interactions, such as due to hydrogen bonding or other van der Walls forces. The force to break a covalent bond is high, e.g., about 1500 pN for a carbon to carbon bond. The force to break a combination of strong protein interactions is typically a magnitude less, e.g., biotin to streptavidin is about 150 pN. Thus, a skilled artisan would understand that conjugation must be strong enough to bind molecular entities in order to implement the intended results.

“Subject” refers to any animal, preferably a human patient, livestock, rodent, monkey, or domestic pet. The term is used herein to encompasses apparently healthy, non-infected individuals or a patient who is known to be infected with, diagnosed with, a pathogen.

The term “comprising” in reference to a peptide having an amino acid sequence refers a peptide that may contain additional N-terminal (amine end) or C-terminal (carboxylic acid end) amino acids, i.e., the term is intended to include the amino acid sequence within a larger peptide. The term “consisting of” in reference to a peptide having an amino acid sequence refers a peptide having the exact number of amino acids in the sequence and not more or having not more than a rage of amino acids expressly specified in the claim. In certain embodiments, the disclosure contemplates that the “N-terminus of a peptide may consist of an amino acid sequence,” which refers to the N-terminus of the peptide having the exact number of amino acids in the sequence and not more or having not more than a rage of amino acids specified in the claim however the C-terminus may be connected to additional amino acids, e.g., as part of a larger peptide. Similarly, the disclosure contemplates that the “C-terminus of a peptide may consist of an amino acid sequence,” which refers to the C-terminus of the peptide having the exact number of amino acids in the sequence and not more or having not more than a rage of amino acids specified in the claim however the N-terminus may be connected to additional amino acids, e.g., as part of a larger peptide.

“Cancer” refers any of various cellular diseases with malignant neoplasms characterized by the proliferation of cells. It is not intended that the diseased cells must actually invade surrounding tissue and metastasize to new body sites. Cancer can involve any tissue of the body and have many different forms in each body area. Within the context of certain embodiments, whether “cancer is reduced” may be identified by a variety of diagnostic manners known to one skill in the art including, but not limited to, observation the reduction in size or number of tumor masses or if an increase of apoptosis of cancer cells observed, e.g., if more than a 5% increase in apoptosis of cancer cells is observed for a sample compound compared to a control without the compound. It may also be identified by a change in relevant biomarker or gene expression profile, such as PSA for prostate cancer, HER2 for breast cancer, or others.

As used herein, the terms “treat” and “treating” are not limited to the case where the subject (e.g., patient) is cured and the disease is eradicated. Rather, embodiments, of the present disclosure also contemplate treatment that merely reduces symptoms, and/or delays disease progression.

As used herein, the term “combination with” when used to describe administration with an additional treatment means that the agent may be administered prior to, together with, or after the additional treatment, or a combination thereof. As used herein, the term “intermixed with” when used to describe administration in combination with an additional treatment means that the agent may be administered “together with.”

The term “effective amount” refers to that amount of a compound or pharmaceutical composition described herein that is sufficient to affect the intended application including, but not limited to, disease treatment, as illustrated below. In relation to a combination therapy, an “effective amount” indicates the combination of agent results in synergistic or additive effect when compared to the agents individually. The therapeutically effective amount can vary depending upon the intended application (in vitro or in vivo), or the subject and disease condition being treated, e.g., the weight and age of the subject, the severity of the disease condition, the manner of administration and the like, which can readily be determined by one of ordinary skill in the art. The specific dose will vary depending on, for example, the particular compounds chosen, the dosing regimen to be followed, whether it is administered in combination with other agents, timing of administration, the tissue to which it is administered, and the physical delivery system in which it is carried.

In certain embodiments, the disclosure relates to recombinant polypeptides comprising sequences disclosed herein or variants or fusions thereof wherein the amino terminal end or the carbon terminal end of the amino acid sequence is optionally attached to a heterologous amino acid sequence, label, or reporter molecule.

A “label” refers to a detectable compound or composition that is conjugated directly or indirectly to another molecule, such as an antibody or a protein, to facilitate detection of that molecule. Specific, non-limiting examples of labels include fluorescent tags, enzymatic linkages, and radioactive isotopes. A label includes the incorporation of a radiolabeled amino acid or the covalent attachment of biotinyl moieties to a polypeptide that can be detected by marked avidin (for example, streptavidin containing a fluorescent marker or enzymatic activity that can be detected by optical or colorimetric methods). Various methods of labeling polypeptides and glycoproteins are known in the art and may be used. Examples of labels for polypeptides include, but are not limited to, the following: radioisotopes or radionucleotides (such as ³⁵S or ¹³¹I) fluorescent labels (such as fluorescein isothiocyanate (FITC), rhodamine, lanthanide phosphors), enzymatic labels (such as horseradish peroxidase, beta-galactosidase, luciferase, alkaline phosphatase), chemiluminescent markers, biotinyl groups, predetermined polypeptide epitopes recognized by a secondary reporter (such as a leucine zipper pair sequences, binding sites for secondary antibodies, metal binding domains, epitope tags), or magnetic agents, such as gadolinium chelates. In some embodiments, labels are attached by spacer arms of various lengths to reduce potential steric hindrance.

In certain embodiments, this disclosure contemplates that chimeric proteins disclosed herein may be variants. Variants may include 1 or 2 amino acid substitutions or conserved substitutions. Variants may include 3 or 4 amino acid substitutions or conserved substitutions. Variants may include 5 or 6 or more amino acid substitutions or conserved substitutions. Variants include those with not more than 1% or 2% of the amino acids are substituted. Variants include those with not more than 3% or 4% of the amino acids are substituted. Variants include proteins with greater than 80%, 89%, 90%, 95%, 98%, or 99% identity or similarity.

Variant peptides can be produced by mutating a vector to produce appropriate codon alternatives for polypeptide translation. Active variants and fragments can be identified with a high probability using computer modeling. Shihab et al. report an online genome tolerance browser. BMC Bioinformatics, 2017, 18 (1): 20. Ng et al. report methods of predicting the effects of amino acid substitutions on protein function. Annu Rev Genomics Hum Genet, 2006, 7:61-80. Teng et al. Approaches and resources for prediction of the effects of non-synonymous single nucleotide polymorphism on protein function and interactions. Curr Pharm Biotechnol, 2008, 9 (2): 123-33.

Desired amino acid substitutions (whether conservative or non-conservative) can be determined by those skilled in the art at the time such substitutions are desired. Guidance in determining which and how many amino acid residues may be substituted, inserted or deleted without abolishing biological activity may be found using computer programs well known in the art, for example, RaptorX, ESyPred3D, HHpred, Homology Modeling Professional for HyperChem, DNAStar, SPARKS-X, EVfold, Phyre, and Phyre2 software. See also Saldano et al. Evolutionary Conserved Positions Define Protein Conformational Diversity, PLOS Comput Biol. 2016, 12 (3): e1004775; Marks et al. Protein structure from sequence variation, Nat Biotechnol. 2012, 30 (11): 1072-80; Mackenzie et al. Curr Opin Struct Biol, 2017, 44:161-167 Mackenzie et al. Proc Natl Acad Sci U S A. 113 (47): E7438-E7447 (2016); Joseph et al. J R Soc Interface, 2014, 11 (95): 20131147, Wei et al. Int. J. Mol. Sci. 2016, 17 (12), 2118. Variants can be tested in functional assays. Certain variants have less than 10%, and preferably less than 5%, and still more preferably less than 2% changes (whether substitutions, deletions, and so on).

Sequence “identity” refers to the number of exactly matching amino acids (expressed as a percentage) in a sequence alignment between two sequences of the alignment calculated using the number of identical positions divided by the greater of the shortest sequence or the number of equivalent positions excluding overhangs wherein internal gaps are counted as an equivalent position. In certain embodiments, any recitation of sequence identity expressed herein may be substituted for sequence similarity. Percent “similarity” is used to quantify the similarity between two sequences of the alignment. This method is identical to determining the identity except that certain amino acids do not have to be identical to have a match. Amino acids are classified as matches if they are among a group with similar properties according to the following amino acid groups: Aromatic—F Y W; hydrophobic—A V I L; Charged positive: R K H; Charged negative—D E; Polar—S T N Q. The amino acid groups are also considered conserved substitutions.

Percent identity can be determined, for example, by comparing sequence information using the GAP computer program, version 6.0, available from the University of Wisconsin Genetics Computer Group (UWGCG). The GAP program utilizes the alignment method of Needleman and Wunsch (J Mol Biol 1970 48:443), as revised by Smith and Waterman (Adv Appl Math 1981 2:482). Briefly, the GAP program defines identity as the number of aligned symbols (i.e., nucleotides or amino acids) which are identical, divided by the total number of symbols in the shorter of the two sequences. The preferred default parameters for the GAP program include: (1) a unitary comparison matrix (containing a value of 1 for identities and 0 for non-identities) and the weighted comparison matrix of Gribskov and Burgess (Nucl Acids Res 1986 14:6745), as described by Schwartz and Dayhoff (eds., Atlas of Protein Sequence and Structure, National Biomedical Research Foundation, Washington, D.C. 1979, pp. 353-358); (2) a penalty of 3.0 for each gap and an additional 0.10 penalty for each symbol in each gap; and (3) no penalty for end gaps.

The terms “expression vector” refer to a recombinant nucleic acid containing a desired coding sequence and appropriate nucleic acid sequences necessary for the expression of the operably linked coding sequence in a particular host organism or expression system, e.g., cellular or cell-free. Nucleic acid sequences necessary for expression in prokaryotes usually include a promoter, an operator (optional), and a ribosome binding site, often along with other sequences. Eukaryotic cells are known to utilize promoters, enhancers, and termination and polyadenylation signals.

In certain embodiments, a vector optionally comprises a gene vector element (nucleic acid) such as a selectable marker region, lac operon, a CMV promoter, a hybrid chicken B-actin/CMV enhancer (CAG) promoter, tac promoter, T7 RNA polymerase promoter, SP6 RNA polymerase promoter, SV40 promoter, internal ribosome entry site (IRES) sequence, cis-acting woodchuck post regulatory element (WPRE), scaffold-attachment region (SAR), inverted terminal repeats (ITR), FLAG tag coding region, c-myc tag coding region, metal affinity tag coding region, streptavidin binding peptide tag coding region, polyHis tag coding region, HA tag coding region, MBP tag coding region, GST tag coding region, polyadenylation coding region, SV40 polyadenylation signal, SV40 origin of replication, Col E1 origin of replication, f1 origin, pBR322 origin, or pUC origin, TEV protease recognition site, loxP site, Cre recombinase coding region, or a multiple cloning site such as having 5, 6, or 7 or more restriction sites within a continuous segment of less than 50 or 60 nucleotides or having 3 or 4 or more restriction sites with a continuous segment of less than 20 or 30 nucleotides.

Protein “expression systems” refer to in vivo and in vitro (cell free) systems. Systems for recombinant protein expression typically utilize somatic cells transfected with a DNA expression vector that contains the template. The cells are cultured under conditions such that they translate the desired protein. Expressed proteins are extracted for subsequent purification. In vivo protein expression systems using prokaryotic and eukaryotic cells are well known. Also, some proteins are recovered using denaturants and protein-refolding procedures. In vitro (cell-free) protein expression systems typically use translation-compatible extracts of whole cells or compositions that contain components sufficient for transcription, translation and optionally post-translational modifications such as RNA polymerase, regulatory protein factors, transcription factors, ribosomes, tRNA cofactors, amino acids and nucleotides. In the presence of an expression vector, these extracts and components can synthesize proteins of interest. Cell-free systems typically do not contain proteases and enable labelling of the protein with modified amino acids. Some cell free systems incorporated encoded components for translation into the expression vector. See, e.g., Shimizu et al., Cell-free translation reconstituted with purified components, 2001, Nat. Biotechnol., 19, 751-755 and Asahara & Chong, Nucleic Acids Research, 2010, 38 (13): e141, both hereby incorporated by reference in their entirety.

A “selectable marker” is a nucleic acid introduced into a vector that encodes a polypeptide that confers a trait suitable for artificial selection or identification (report gene), e.g., beta-lactamase confers antibiotic resistance, which allows an organism expressing beta-lactamase to survive in the presence antibiotic in a growth medium. Another example is thymidine kinase, which makes the host sensitive to ganciclovir selection. It may be a screenable marker that allows one to distinguish between wanted and unwanted cells based on the presence or absence of an expected color. For example, the lac-z-gene produces a beta-galactosidase enzyme which confers a blue color in the presence of X-gal (5-bromo-4-chloro-3-indolyl-β-D-galactoside). If recombinant insertion inactivates the lac-z-gene, then the resulting colonies are colorless. There may be one or more selectable markers, e.g., an enzyme that can complement to the inability of an expression organism to synthesize a particular compound required for its growth (auxotrophic) and one able to convert a compound to another that is toxic for growth. URA3, an orotidine-5′ phosphate decarboxylase, is necessary for uracil biosynthesis and can complement ura3 mutants that are auxotrophic for uracil. URA3 also converts 5-fluoroorotic acid into the toxic compound 5-fluorouracil. Additional contemplated selectable markers include any genes that impart antibacterial resistance or express a fluorescent protein. Examples include, but are not limited to, the following genes: ampr, camr, tetr, blasticidinr, neor, hygr, abxr, neomycin phosphotransferase type II gene (nptII), p-glucuronidase (gus), green fluorescent protein (gfp), egfp, yfp, mCherry, p-galactosidase (lacZ), lacZa, lacZAM15, chloramphenicol acetyltransferase (cat), alkaline phosphatase (phoA), bacterial luciferase (luxAB), bialaphos resistance gene (bar), phosphomannose isomerase (pmi), xylose isomerase (xylA), arabitol dehydrogenase (atlD), UDP-glucose: galactose-1-phosphate uridyltransferasel (galT), feedback-insensitive α subunit of anthranilate synthase (OASAID), 2-deoxyglucose (2-DOGR), benzyladenine-N-3-glucuronide, E. coli threonine deaminase, glutamate 1-semialdehyde aminotransferase (GSA-AT), D-amino acidoxidase (DAAO), salt-tolerance gene (rstB), ferredoxin-like protein (pflp), trehalose-6-P synthase gene (AtTPS1), lysine racemase (lyr), dihydrodipicolinate synthase (dapA), tryptophan synthase beta 1 (AtTSB1), dehalogenase (dhlA), mannose-6-phosphate reductase gene (M6PR), hygromycin phosphotransferase (HPT), and D-serine ammonialyase (dsdA).

### T Cell Receptor (TCR), and Functional Portions and Functional Variants Thereof

In certain embodiments, this disclosure relates to a T cell receptor (TCR), and functional portions and functional variants thereof, having specificity for human papillomavirus (HPV) 16 epitopes disclosed herein. In certain embodiments, the TCR has specificity for HPV 16 E2, E5 and E6 amino acid segments disclosed herein, e.g., as presented on HLAs. The HPV 16 is a subtype of HPV that is most commonly associated with malignancy.

The TCR may have antigenic specificity for any HPV 16 E2, E5, or E6 protein, polypeptide, or peptide. In certain embodiments, the TCR has antigenic specificity for an HPV 16 protein comprising, consisting of, or consisting essentially of, the amino acid sequences of SEQ ID NO: 1-9.

It is contemplated that TCRs are able to recognize HPV 16 proteins in a major histocompatibility complex (MHC) class I-dependent manner. “MHC class I-dependent manner,” as used herein, means that the TCR elicits an immune response upon binding to HPV 16 protein within the context of an MHC class I molecule. The MHC class I molecule can be any MHC class I molecule known in the art, e.g., HLA-A molecules.

In certain embodiments, this disclosure relates to cells comprising recombination vectors encoding TCR sequences disclosed herein. In certain embodiments, T cells are isolated or groups of cells that contain T cells, e.g., mononuclear cells or leukocytes. The cells are optionally expanded, exposed to a recombinant vector for expression of the TCR sequences disclosed herein for expression on the cell surface. The TCR expressing cells are then administered to a subject in need of a treatment due to an HPV infection or related cancer.

Whole blood is composed of plasma, red blood cells (RBCs; or erythrocytes), platelets, and nucleated white blood cells, also referred to as leukocytes. The leukocytes can be further categorized into mononuclear cells and polymorphonuclear cells (or granulocytes). Different techniques to obtain peripheral blood mononuclear cells (PBMCs), polymorphonuclear cells, leukocytes, or specific cell subsets, e.g., isolate specific cells directly by using flow cytometry, depleting red blood cells, centrifugation, apheresis.

The TCRs, including functional portions and functional variants thereof, provide advantages, including when expressed by cells used for adoptive cell transfer. Without being bound by a particular theory or mechanism, it is believed that because HPV 16 protein is expressed by HPV 16-infected cells of multiple cancer types, the TCRs, including functional portions and functional variants thereof, provide the ability to destroy cells of multiple types of HPV 16-associated cancer and, accordingly, treat or prevent multiple types of HPV 16-associated cancer. Additionally, without being bound to a particular theory or mechanism, it is believed that because the HPV 16 protein is expressed primarily in cancer cells, HPV 16-infected cells, or HPV-positive premalignancy cells, the TCRs, including functional portions and functional variants thereof, target the destruction of cancer cells, HPV 16-infected cells, or HPV-positive premalignancy cells, while minimizing or eliminating the destruction of normal, non-cancerous, non-HPV-infected, and non-HPV-positive premalignant cells, thereby reducing, for example, by minimizing or eliminating, toxicity. Moreover, the TCRs may successfully treat or prevent HPV-positive cancers that do not respond to other types of treatment such as, for example, chemotherapy alone, surgery, or radiation. Additionally, the TCRs provide highly avid recognition of HPV which may provide the ability to recognize unmanipulated tumor cells (e.g., tumor cells that have not been treated with interferon-gamma, transfected with a vector encoding one or both of HPV 16 proteins and HLA, pulsed with the epitope peptide, or a combination thereof).

The terms “specifically binds” or “specificity,” as used herein, refer to a TCR that can specifically bind to and immunologically recognize an HPV 16 protein (e.g., E2, E5, and E6) with preference over other proteins generally. Antigenic specificity also has avidity that causes release of IFN-gamma from the T cell. For example, a TCR may be considered to have “specificity” for HPV 16 if T cells expressing the TCR secrete at least about 200 pg/mL or more (e.g., 200 pg/mL or more, 300 pg/mL or more, 400 pg/mL or more, 500 pg/mL or more, 600 pg/mL or more, 700 pg/mL or more, 1000 pg/mL or more, 5,000 pg/mL or more, 7,000 pg/mL or more, 10,000 pg/mL or more, or 20,000 pg/mL or more) of interferon gamma (IFN-gamma) upon co-culture with antigen-negative HLA target cells pulsed with a low concentration of HPV 16 peptide (e.g., about 0.05 ng/mL to about 5 ng/ml, 0.05 ng/mL, 0.1 ng/mL, 0.5 ng/mL, 1 ng/ml, or 5 ng/mL). Alternatively or additionally, a TCR may be considered to have “antigenic specificity” for a HPV 16 protein if T cells expressing the TCR secrete at least twice as much IFN-gamma as the untransduced peripheral blood lymphocyte (PBL) background level of IFN-gamma upon co-culture with antigen-negative HLA target cells pulsed with a low concentration of the HPV 16 peptide. Cells expressing the TCRs, including functional portions and functional variants thereof, may also secrete IFN-gamma upon co-culture with antigen-negative HLA target cells pulsed with higher concentrations of the HPV 16 peptide.

In certain embodiments, TCR comprising two polypeptides (i.e., polypeptide chains), such as an alpha chain of a TCR, a beta chain of a TCR, a gamma chain of a TCR, a delta chain of a TCR, or a combination thereof. The polypeptides of the TCR can comprise any amino acid sequence, provided that the TCR has specificity for an HPV 16 protein.

In certain embodiments, this disclosure relates to a cell comprising a T cell receptor (TCR) having antigenic specificity for human papillomavirus (HPV) protein E2, E5, or E6 and comprising a human variable region and a heterologous constant region. In certain embodiments, the TCR is isolated or purified and has antigenic specificity for HPV epitope disclosed herein, e.g., the amino acid sequences of SEQ ID NOs: 1-9.

In certain embodiments, this disclosure relates to a T cell receptor (TCR) having antigenic specificity for human papillomavirus (HPV) E2, E5, or E6 and comprising a human variable region and a constant region. In certain embodiments, the TCR alpha chain and beta chain comprise the amino acid sequences as disclosed herein or variants thereof.

In certain embodiments, the T cell receptor (TCR) has a variant or chimeric sequence that does not naturally occur. In certain embodiments, the variable region is a variant or chimeric sequence that does not naturally occur. In certain embodiments, the constant region is a variant or chimeric sequence that does not naturally occur.

In certain embodiments, the T cell receptor (TCR) or nucleic acid encoding the TCR or protein has a variant or chimeric sequence that does not naturally occur. In certain embodiments, the variable region is a variant or chimeric sequence that does not naturally occur. In certain embodiments, the constant region is a variant or chimeric sequence that does not naturally occur. In certain embodiments, the nucleic acid comprises a codon optimized sequence that does not naturally occur. In certain embodiments, a nucleic acid comprises synonymous or non-synonymous codon substitutions such that the nucleic acid does not naturally occur or the encoded TCR or polypeptide does not naturally occur.

In certain embodiments, this disclosure relates to a T cell receptor (TCR) having specificity for human papillomavirus (HPV) E2, E5, or E6 and comprising a human variable region and a constant region, wherein the TCR comprises the alpha chain complementarity determining region the alpha chain CDR3 as disclosed herein and the beta chain CDR3 as disclosed herein. In certain embodiments, the TCR alpha chain and beta chain comprise the amino acid sequences as disclosed herein or variants thereof.

In certain embodiments, this disclosure relates to a T cell receptor (TCR) having specificity for human papillomavirus (HPV) E2, E5, or E6 and comprising a human variable region and a heterologous constant region, wherein the TCR comprises the alpha chain complementarity determining region the alpha chain CDR3 as disclosed herein and the beta chain CDR3 as disclosed herein. In certain embodiments, the TCR alpha chain and beta chain comprise the amino acid sequences as disclosed herein or variants thereof.

In certain embodiments, this disclosure relates to a T cell receptor (TCR) having specificity for human papillomavirus (HPV) E2, E5, or E6 and comprising a human variable region and a constant region, wherein the TCR comprises the alpha chain complementarity determining region CDR 1 as disclosed herein, the alpha chain CDR2 as disclosed herein, the alpha chain CDR3 as disclosed herein, the beta chain CDRI as disclosed herein, the beta chain CDR2 as disclosed herein, and the beta chain CDR3 as disclosed herein. In certain embodiments, the TCR alpha chain and beta chain comprise the amino acid sequences as disclosed herein.

In certain embodiments, this disclosure relates to a T cell receptor (TCR) having specificity for human papillomavirus (HPV) E2, E5, or E6 and comprising a human variable region and a heterologous constant region, wherein the TCR comprises the alpha chain complementarity determining region CDR 1 as disclosed herein, the alpha chain CDR2 as disclosed herein, the alpha chain CDR3 as disclosed herein, the beta chain CDRI as disclosed herein, the beta chain CDR2 as disclosed herein, and the beta chain CDR3 as disclosed herein. In certain embodiments, the TCR alpha chain and beta chain comprise the amino acid sequences as disclosed herein.

In certain embodiments, the TCR comprises two polypeptide chains, each of which comprises a human variable region comprising a complementarity determining region (CDR) 1, a CDR2, and a CDR3 of a TCR. In this regard, the TCR can comprise any one or more of the amino acid sequences disclosed herein.

In certain embodiments, the TCR can comprise an amino acid sequence of a variable region of a TCR comprising the CDRs and comprise a constant region that is heterologous derived from any suitable species such as, e.g., human or mouse.

In certain embodiments, a functional portion can be any portion comprising contiguous amino acids of the TCR (or functional variant thereof) of which it is a part, provided that the functional portion specifically binds to an HPV 16 protein. The term “functional portion” when used in reference to a TCR (or functional variant thereof) refers to any part or fragment of the TCR (or functional variant thereof), which part or fragment retains the biological activity of the TCR (or functional variant thereof) of which it is a part (the parent TCR or parent functional variant thereof). Functional portions encompass, for example, those parts of a TCR (or functional variant thereof) that retain the ability to specifically bind to a HPV 16 protein (e.g., in an HLA dependent manner), or detect, treat, or prevent cancer, to a similar extent, the same extent, or to a higher extent, as the parent TCR (or functional variant thereof). In reference to the parent TCR (or functional variant thereof), the functional portion can comprise, for instance, about 10%, 25%, 30%, 50%, 68%, 80%, 90%, 95%, or more, of the parent TCR (or functional variant thereof).

The functional portion can comprise additional amino acids at the amino or carboxy terminus of the portion, or at both termini, which additional amino acids are not found in the amino acid sequence of the parent TCR or functional variant thereof. Desirably, the additional amino acids do not interfere with the biological function of the functional portion, e.g., specifically binding to an HPV 16 protein disclosed herein; and/or having the ability to detect cancer, treat or prevent cancer, etc. More desirably, the additional amino acids enhance the biological activity, as compared to the biological activity of the parent TCR or functional variant thereof.

In certain embodiments, this disclosure relates to a fusion protein comprising at least one of the polypeptides described herein along with at least one other polypeptide. The other polypeptide can exist as a separate polypeptide of the fusion protein, or can exist as a polypeptide, which is expressed in frame (in tandem) with one of the polypeptides described herein. The other polypeptide can encode any peptidic or proteinaceous molecule, or a portion thereof, including, but not limited to an immunoglobulin, CD3, CD4, CD8, an MHC molecule, a CDI molecule, e.g., CDla, CD1b, CD1c, CD1d, etc.

A fusion protein of this disclosure can comprise one or more copies of the polypeptide and/or one or more copies of the other polypeptide. For instance, the fusion protein can comprise 1, 2, 3, 4, 5, or more, copies of the polypeptide and/or of the other polypeptide. Suitable methods of making fusion proteins are known in the art, and include, for example, recombinant methods. See, for instance, Choi et al., Mol. Biotechnol. 31:193-202 (2005).

In certain embodiments, the TCRs (and functional portions and functional variants thereof), polypeptides, and proteins may be expressed as a single protein comprising a linker peptide linking the alpha chain and the beta chain. In this regard, the TCRs (and functional variants and functional portions thereof), polypeptides, and proteins of this disclosure comprising the amino acid sequences may further comprise a linker peptide. The linker peptide may advantageously facilitate the expression of a recombinant TCR (including functional portions and functional variants thereof), polypeptide, and/or protein in a host cell. The linker peptide may comprise any suitable amino acid sequence. For example, the linker peptide may comprise the amino acids glycine, polyglycine, serine, alanine, proline, or combinations thereof. Upon expression of the construct including the linker peptide by a host cell, the linker peptide may be cleaved, resulting in separated alpha and beta chains.

In certain embodiments, this disclosure relates to a recombinant antibody comprising at least one of the TCR polypeptides described herein. As used herein, “recombinant antibody” refers to a recombinant (e.g., genetically engineered) protein comprising at least one of the polypeptides disclosed herein and a polypeptide chain of an antibody, or a portion thereof. The polypeptide of an antibody, or portion thereof, can be a heavy chain, a light chain, a variable or constant region of a heavy or light chain, a single chain variable fragment (scFv), or an Fc, Fab, or F (ab)₂′ fragment of an antibody, etc. The polypeptide chain of an antibody, or portion thereof, can exist as a separate polypeptide of the recombinant antibody. Alternatively, the polypeptide chain of an antibody, or portion thereof, can exist as a polypeptide, which is expressed in frame (in tandem) with the polypeptide. The polypeptide of an antibody, or portion thereof, can be a polypeptide of any antibody or any antibody fragment, including any of the antibodies and antibody fragments described herein.

In certain embodiments, this disclosure contemplates functional variants of the TCRs described herein. The term “functional variant” as used herein refers to a TCR, polypeptide, or protein having substantial or significant sequence identity or similarity to a parent TCR, polypeptide, or protein, which functional variant retains the biological activity of the TCR, polypeptide, or protein of which it is a variant. Functional variants encompass, for example, those variants of the TCR, polypeptide, or protein described herein (the parent TCR, polypeptide, or protein) that retain the ability to specifically bind to HPV 16 protein for which the parent TCR has antigenic specificity or to which the parent polypeptide or protein specifically binds, to a similar extent, the same extent, or to a higher extent, as the parent TCR, polypeptide, or protein. In reference to the parent TCR, polypeptide, or protein, the functional variant can, for instance, be at least about 30%, 50%, 75%, 80%, 90%, 95%, 96%, 97%, 98%, 99% or more identical in amino acid sequence to the parent TCR, polypeptide, or protein.

The functional variant can, for example, comprise the amino acid sequence of the parent

TCR, polypeptide, or protein with at least one conservative amino acid substitution. Conservative amino acid substitutions are known in the art and include amino acid substitutions in which one amino acid having certain physical and/or chemical properties is exchanged for another amino acid that has the same chemical or physical properties. For instance, the conservative amino acid substitution can be an acidic amino acid substituted for another acidic amino acid (e.g., Asp or Glu), an amino acid with a nonpolar side chain substituted for another amino acid with a nonpolar side chain (e.g., Ala, Gly, Val, Ile, Leu, Met, Phe, Pro, Trp, Val, etc.), a basic amino acid substituted for another basic amino acid (Lys, Arg, etc.), an amino acid with a polar side chain substituted for another amino acid with a polar side chain (Asn, Cys, Gln, Ser, Thr, Tyr, etc.), etc.

Alternatively or additionally, the functional variants can comprise the amino acid sequence of the parent TCR, polypeptide, or protein with at least one non-conservative amino acid substitution. In this case, it is preferable for the non-conservative amino acid substitution to not interfere with or inhibit the biological activity of the functional variant. Preferably, the non-conservative amino acid substitution enhances the biological activity of the functional variant, such that the biological activity of the functional variant is increased as compared to the parent TCR, polypeptide, or protein.

The TCRs, polypeptides, and proteins disclosed herein (including functional variants thereof) can be of any length, i.e., can comprise any number of amino acids, provided that the TCRs, polypeptides, or proteins (or functional variants thereof) retain their biological activity, e.g., the ability to specifically bind to HPV 16 E6; detect cancer, HPV 16 infection, or HPV-positive premalignancy in a mammal; or treat or prevent cancer, HPV 16 infection, or HPV-positive premalignancy in a mammal, etc. For example, the polypeptide can be in the range of from about 50 to about 5000 amino acids long, such as 50, 70, 75, 100, 125, 150, 175, 200, 300, 400, 500, 600, 700, 800, 900, 1000 or more amino acids in length.

The TCRs, polypeptides, and proteins disclosed here (including functional variants thereof) can comprise synthetic amino acids in place of one or more naturally-occurring amino acids. Such synthetic amino acids are known in the art, and include, for example, aminocyclohexane carboxylic acid, norleucine, alpha-amino n-decanoic acid, homoserine, S-acetylaminomethyl-cysteine, trans-3- and trans-4-hydroxyproline, 4-aminophenylalanine, 4-nitrophenylalanine, 4-chlorophenylalanine, 4-carboxyphenylalanine, beta-phenylserine beta-hydroxyphenylalanine, phenylglycine, alpha-naphthylalanine, cyclohexylalanine, cyclohexylglycine, indoline-2-carboxylic acid, 1,2,3,4-tetrahydroisoquinoline-3-carboxylic acid, aminomalonic acid, aminomalonic acid monoamide, N′-benzyl-N′-methyl-lysine, N′,N′-dibenzyl-lysine, 6-hydroxylysine, ornithine, alpha-aminocyclopentane carboxylic acid, alpha-aminocyclohexane carboxylic acid, alpha-aminocycloheptane carboxylic acid, alpha-(2-amino-2-norbornane)-carboxylic acid, alpha, gamma-diaminobutyric acid, alpha,beta-diaminopropionic acid, homophenylalanine, and alpha-tert-butylglycine.

The TCRs, polypeptides, and proteins disclosed herein (including functional variants thereof) can be glycosylated, amidated, carboxylated, phosphorylated, esterified, N-acylated, cyclized via, e.g., a disulfide bridge, or converted into an acid addition salt and/or optionally dimerized or polymerized, or conjugated.

In certain embodiments, nucleic acids can contain natural, non-natural, or altered nucleotides, and which can contain a natural, non-natural or altered internucleotide linkage, such as a phosphoramidate linkage or a phosphorothioate linkage, instead of the phosphodiester found between the nucleotides of an unmodified oligonucleotide. In an embodiment, the nucleic acid comprises complementary DNA (cDNA).

In certain embodiments, a nucleic acid comprising a nucleotide sequence which is complementary to the nucleotide sequence of any of the nucleic acids described herein or a nucleotide sequence which hybridizes under stringent conditions to the nucleotide sequence of any of the nucleic acids described herein. The nucleotide sequence which hybridizes under stringent conditions preferably hybridizes under high stringency conditions. By “high stringency conditions” is meant that the nucleotide sequence specifically hybridizes to a target sequence (the nucleotide sequence of any of the nucleic acids described herein) in an amount that is detectably stronger than non-specific hybridization. High stringency conditions include conditions which would distinguish a polynucleotide with an exact complementary sequence, or one containing only a few scattered mismatches from a random sequence that happened to have a few small regions (e.g., 3-10 bases) that matched the nucleotide sequence. Such small regions of complementarity are more easily melted than a full-length complement of 14-17 or more bases, and high stringency hybridization makes them easily distinguishable. Relatively high stringency conditions would include, for example, low salt and/or high temperature conditions, such as provided by about 0.02-0.1 M NaCl or the equivalent, at temperatures of about 50-70 degrees C. Such high stringency conditions tolerate little, if any, mismatch between the nucleotide sequence and the template or target strand and are suitable for detecting expression of any of the TCRs (including functional portions and functional variants thereof). It is generally appreciated that conditions can be rendered more stringent by the addition of increasing amounts of formamide.

In certain embodiments, nucleic acids encoding a peptide can be incorporated into a recombinant expression vector. In this regard, the recombinant expression vector comprises a nucleotide sequence encoding the alpha chain, the beta chain, and linker peptide. For example, in an embodiment, the recombinant expression vector comprises a codon-optimized nucleotide sequence comprising encoding chimeric alpha and beta chains with a linker positioned between them.

In certain embodiments, recombinant expression vector permits the expression of an mRNA, protein, polypeptide, or peptide by a host cell, when the construct comprises a nucleotide sequence encoding the mRNA, protein, polypeptide, or peptide, and the vector is contacted with the cell under conditions sufficient to have the mRNA, protein, polypeptide, or peptide expressed within the cell. The vectors are not naturally occurring as a whole. However, parts of the vectors can be naturally occurring. The recombinant expression vectors can comprise any type of nucleotides, including, but not limited to DNA and RNA, which can be single-stranded or double-stranded, synthesized or obtained in part from natural sources, and which can contain natural, non-natural, or altered nucleotides. The recombinant expression vectors can comprise naturally occurring, non-naturally occurring internucleotide linkages, or both types of linkages. Preferably, the non-naturally occurring or altered nucleotides or internucleotide linkages does not hinder the transcription or replication of the vector. The recombinant expression vector can be any suitable recombinant expression vector and can be used to transform or transfect any suitable host cell. Suitable vectors include those designed for propagation and expansion or for expression or both, such as plasmids and viruses.

In certain embodiments, the recombinant expression vector comprises regulatory sequences, such as transcription and translation initiation and termination codons, which are specific to the type of host cell (e.g., bacterium, fungus, plant, or animal) into which the vector is to be introduced, as appropriate and taking into consideration whether the vector is DNA- or RNA-based.

The recombinant expression vector can include one or more marker genes, which allow for selection of transformed or transfected host cells. Marker genes include biocide resistance, e.g., resistance to antibiotics, heavy metals, etc., complementation in an auxotrophic host cell to provide prototrophy, and the like. Suitable marker genes for the expression vectors include, for instance, neomycin/G418 resistance genes, hygromycin resistance genes, histidinol resistance genes, tetracycline resistance genes, and ampicillin resistance genes.

The recombinant expression vector can comprise a native or nonnative promoter operably linked to the nucleotide sequence encoding the TCR, polypeptide, or protein (including functional variants thereof), or to the nucleotide sequence which is complementary to or which hybridizes to the nucleotide sequence encoding the TCR, polypeptide, or protein (including functional variants thereof). The selection of promoters, e.g., strong, weak, inducible, tissue-specific, and developmental-specific, is within the ordinary skill of the artisan. Similarly, the combining of a nucleotide sequence with a promoter is also within the skill of the artisan. The promoter can be a non-viral promoter or a viral promoter, e.g., a cytomegalovirus (CMV) promoter, an SV40 promoter, an RSV promoter, and a promoter found in the long-terminal repeat of the murine stem cell virus.

The recombinant expression vectors can be designed for either transient expression, for stable expression, or for both. Also, the recombinant expression vectors can be made for constitutive expression or for inducible expression. Further, the recombinant expression vectors can be made to include a suicide gene. As used herein, the term “suicide gene” refers to a gene that causes the cell expressing the suicide gene to die. The suicide gene can be a gene that confers sensitivity to an agent, e.g., a drug, upon the cell in which the gene is expressed, and causes the cell to die when the cell is contacted with or exposed to the agent. Suicide genes are known in the art (see, for example, Suicide Gene Therapy: Methods and Reviews, Springer, Caroline J. (Cancer Research UK Centre for Cancer Therapeutics at the Institute of Cancer Research, Sutton, Surrey, UK), Humana Press, 2004) and include, for example, the Herpes Simplex Virus (HSV) thymidine kinase (TK) gene, cytosine deaminase, purine nucleoside phosphorylase, and nitroreductase.

In certain embodiments, this disclosure relates to a host cell comprising any of the recombinant expression vectors described herein. As used herein, the term “host cell” refers to any type of cell that can contain the recombinant expression vector. The host cell can be a eukaryotic cell, e.g., plant, animal, fungi, or algae, or can be a prokaryotic cell, e.g., bacteria or protozoa. The host cell can be a cultured cell or a primary cell, i.e., isolated directly from an organism, e.g., a human. The host cell can be an adherent cell or a suspended cell, i.e., a cell that grows in suspension. Suitable host cells are known in the art and include, for instance, DH5alpha E. coli cells, Chinese hamster ovarian cells, monkey VERO cells, COS cells, HEK293 cells, and the like. For purposes of amplifying or replicating the recombinant expression vector, the host cell is preferably a prokaryotic cell, e.g., a DH5alpha, cell. For purposes of producing a recombinant TCR, polypeptide, or protein, the host cell is preferably a mammalian cell. Most preferably, the host cell is a human cell. While the host cell can be of any cell type, can originate from any type of tissue, and can be of any developmental stage, the host cell preferably is a peripheral blood lymphocyte (PBL) or a peripheral blood mononuclear cell (PBMC). More preferably, the host cell is a T cell.

For purposes herein, the T cell can be any T cell, such as a cultured T cell, e.g., a primary T cell, or a T cell from a cultured T cell line, e.g., Jurkat, SupTI, etc., or a T cell obtained from a mammal. If obtained from a mammal, the T cell can be obtained from numerous sources, including but not limited to blood, bone marrow, lymph node, the thymus, or other tissues or fluids. T cells can also be enriched for or purified. Preferably, the T cell is a human T cell. More preferably, the T cell is a T cell isolated from a human. The T cell can be any type of T cell and can be of any developmental stage, including but not limited to, CD4+//CD8+ double positive T cells, CD4+ helper T cells, e.g., Th1 and Th2 cells, CD4+ T cells, CD8+ T cells (e.g., cytotoxic T cells), tumor infiltrating lymphocytes (TILs), memory T cells (e.g., central memory T cells and effector memory T cells), naive T cells, and the like.

Also provided is a population of cells comprising at least one host cell described herein. The population of cells can be a heterogeneous population comprising the host cell comprising any of the recombinant expression vectors described, in addition to at least one other cell, e.g., a host cell (e.g., a T cell), which does not comprise any of the recombinant expression vectors, or a cell other than a T cell, e.g., a B cell, a macrophage, a neutrophil, an erythrocyte, a hepatocyte, an endothelial cell, an epithelial cells, a muscle cell, a brain cell, etc. Alternatively, the population of cells can be a substantially homogeneous population, in which the population comprises mainly of host cells (e.g., consisting essentially of) comprising the recombinant expression vector. The population also can be a clonal population of cells, in which all cells of the population are clones of a single host cell comprising a recombinant expression vector, such that all cells of the population comprise the recombinant expression vector. In one embodiment, the population of cells is a clonal population comprising host cells comprising a recombinant expression vector as described herein.

### Pharmaceutical Compositions

TCRs, polypeptides, proteins (including functional variants thereof), nucleic acids, recombinant expression vectors, host cells (including populations thereof), particles, and antibodies (including antigen binding portions thereof) can be formulated into a composition, such as a pharmaceutical composition. In this regard, this disclosure contemplates a pharmaceutical composition comprising any of the TCRs, polypeptides, proteins, functional portions, functional variants, nucleic acids, expression vectors, host cells (including populations thereof), particles, and antibodies (including antigen binding portions thereof) described herein, and a pharmaceutically acceptable carrier. This disclosure contemplates pharmaceutical compositions containing any of the TCR materials can comprise more than one polypeptide and a nucleic acid, or two or more different TCRs. Alternatively, the pharmaceutical composition can comprise a TCR material in combination with another pharmaceutically active agent(s) or drug(s), such as a chemotherapeutic agent, e.g., asparaginase, busulfan, carboplatin, cisplatin, daunorubicin, doxorubicin, fluorouracil, gemcitabine, hydroxyurea, methotrexate, paclitaxel, rituximab, vinblastine, vincristine, etc.

Preferably, the carrier is a pharmaceutically acceptable carrier. With respect to pharmaceutical compositions, the carrier can be any of those conventionally used for the particular TCR material under consideration. The choice of carrier will be determined in part by the particular TCR material, as well as by the particular method used to administer the TCR material. Suitable formulations may include any of those for oral, parenteral, subcutaneous, intravenous, intramuscular, intraarterial, intrathecal, or intraperitoneal administration. More than one route can be used to administer the TCR materials, and in certain instances, a particular route can provide a more immediate and more effective response than another route.

Preferably, the TCR material is administered by injection, e.g., intravenously. When the TCR material is a host cell expressing the TCR (or functional variant thereof), the pharmaceutically acceptable carrier for the cells for injection may include any isotonic carrier such as, for example, normal saline (about 0.90% w/v of NaCl in water, about 300 mOsm/L NaCl in water, or about 9.0 g NaCl per liter of water), about 5% dextrose in water, or Ringer's lactate. In an embodiment, the pharmaceutically acceptable carrier is supplemented with human serum albumen.

The amount or dose (e.g., numbers of cells when the TCR material is one or more cells) of the TCR material administered should be sufficient to effect, e.g., a therapeutic or prophylactic response, in the subject or animal over a reasonable time frame. For example, the dose of the TCR material should be sufficient to bind to a cancer antigen, or detect, treat or prevent cancer in a period of from about 2 hours or longer, e.g., 12 to 24 or more hours, 2 days to a week, from the time of administration. In certain embodiments, the time period could be even longer. The dose will be determined by the efficacy of the particular TCR material and the condition of the animal (e.g., human), as well as the body weight of the animal (e.g., human) to be treated.

Assays for determining an administered dose are known in the art. An assay, which comprises comparing the extent to which target cells are lysed or IFN-gamma is secreted by T cells expressing the TCR (or functional variant or functional portion thereof), polypeptide, or protein upon administration of a given dose of such T cells to a mammal among a set of mammals of which is each given a different dose of the T cells, could be used to determine a starting dose to be administered to a mammal. The extent to which target cells are lysed or IFN-gamma is secreted upon administration of a certain dose can be assayed by methods known in the art.

The dose of the TCR material also will be determined by the existence, nature and extent of any adverse side effects that might accompany the administration of a particular TCR material. Typically, the attending physician will decide the dosage of the TCR material with which to treat each individual patient, taking into consideration a variety of factors, such as age, body weight, general health, diet, sex, TCR material to be administered, route of administration, and the severity of the condition being treated. In an embodiment in which the TCR material is a population of cells, the number of cells administered per infusion may vary, e.g., from about 1×10⁶ to about 1×10¹² cells or more.

### Methods of Use

In certain embodiments, this disclosure relates to a method of treating or preventing cancer in a mammal, comprising administering a population of cells to the mammal in an amount effective to treat or prevent the cancer in the mammal, wherein: the population of cells comprises at least one host cell expressing a T-cell receptor (TCR) having antigenic specificity for HPV 16 for epitopes of E2, E5, or E6 as disclosed herein.

A method of treating or preventing a condition in a mammal, comprising administering a population of cells to the mammal in an amount effective to treat or prevent the condition in the mammal, wherein: the population of cells comprises at least one host cell expressing a T-cell receptor (TCR) having antigenic specificity for HPV 16 as disclosed herein wherein the population of cells is allogeneic or autologous to the mammal; and the condition is cancer, HPV 16 infection, or HPV-positive premalignancy.

In certain embodiments, this disclosure relates to an isolated or purified nucleic acid comprising a nucleotide sequence encoding the TCR or protein disclosed herein in operable combination with a heterologous promoter. In certain embodiments, this disclosure relates to a recombinant expression vector comprising the nucleic acid encoding a TCR or protein disclosed herein. In certain embodiments, this disclosure relates to an isolated host cell comprising the recombinant expression vector. In certain embodiments, the host cell according is a human cell. In certain embodiments, this disclosure relates to population of cells comprising at least one host cell.

In certain embodiments, this disclosure relates to a method of treating or preventing a condition in a mammal, the method comprising administering to the mammal a population of host cells comprising a recombinant expression vector comprising a nucleic acid comprising a nucleotide sequence encoding a T cell receptor (TCR) having antigenic specificity for human papillomavirus (HPV) 16 E2, E5, or E6 as disclosed herein wherein the condition is cancer, HPV 16 infection, or HPV-positive premalignancy,

In certain embodiments, this disclosure relates to a pharmaceutical composition comprising the TCR population of cells, and a pharmaceutically acceptable carrier.

In certain embodiments, this disclosure relates to a method of treating or preventing a condition in a mammal, comprising administering the population of cells comprising a TCR as reported herein, e.g., due to expression of a nucleic acid encoding the TCR or functional segment, to the mammal in an amount effective to treat or prevent the condition in the mammal, wherein the condition is cancer, HPV 16 infection, or HPV-positive premalignancy. In certain embodiments, the condition is cancer of the uterine cervix, oropharynx, anus, anal canal, anorectum, vagina, vulva, or penis. In certain embodiments, the condition is an HPV 16-positive cancer.

In certain embodiments, the treatment is administered in combination with another anticancer agent such as cyclophosphamide, aldesleukin, cetuximab, checkpoint inhibitor, anti-PD1 antibody, or anti-PD-L1 antibodies or combinations thereof. In certain embodiment, checkpoint inhibitor is selected from ipilimumab (anti-CTLA-4 antibody), nivolumab, pembrolizumab, and cemiplimab (anti-PD-1 antibodies), atezolizumab, durvalumab, and avelumab (PD-L1).

In certain embodiments, the cancer is acute lymphocytic cancer, acute myeloid leukemia, alveolar rhabdomyosarcoma, bone cancer, brain cancer, breast cancer, cancer of the anus, anal canal, or anorectum, cancer of the eye, cancer of the intrahepatic bile duct, cancer of the joints, cancer of the neck, gallbladder, or pleura, cancer of the nose, nasal cavity, or middle ear, cancer of the oral cavity, cancer of the vagina, cancer of the vulva, chronic lymphocytic leukemia, chronic myeloid cancer, colon cancer, esophageal cancer, uterine cervical cancer, gastrointestinal carcinoid tumor, glioma, Hodgkin lymphoma, hypopharynx cancer, kidney cancer, larynx cancer, liver cancer, lung cancer, malignant mesothelioma, melanoma, multiple myeloma, nasopharynx cancer, non-Hodgkin lymphoma, cancer of the oropharynx, ovarian cancer, cancer of the penis, pancreatic cancer, peritoneum, omentum, and mesentery cancer, pharynx cancer, prostate cancer, rectal cancer, renal cancer, skin cancer, small intestine cancer, soft tissue cancer, stomach cancer, testicular cancer, thyroid cancer, cancer of the uterus, ureter cancer, and urinary bladder cancer.

In certain embodiments, this disclosure relates to a method of preparing a population of HPV-specific T cells, the method comprising: dividing an HPV-positive tumor sample into multiple fragments; separately culturing the multiple fragments; obtaining T cells from the cultured multiple fragments; testing the T cells for HPV antigen recognition of a epitope disclosed herein bound to a MHC; selecting the T cells that exhibit one or both of specific autologous HPV-positive tumor recognition and HPV antigen recognition; and expanding the number of selected T cells to produce a population of HPV-specific T cells.

In certain embodiments, this disclosure relates to a method of treating or preventing cancer in a mammal, the method comprising: dividing an HPV-positive tumor sample into multiple fragments; separately culturing the multiple fragments; obtaining T cells from the cultured multiple fragments; testing the T cells for HPV epitope recognition as reported herein and/or specific autologous HPV-positive tumor recognition; selecting the T cells that exhibit one or both of HPV epitope recognition and specific autologous HPV-positive tumor recognition; expanding the number of selected T cells to produce a population of HPV-specific T cells for adoptive cell therapy; and administering the expanded number of T cells to the mammal in an amount effective to treat or prevent cancer in the mammal. Dividing the tumor sample, culturing the tumor fragments, obtaining T cells, testing the T cells, selecting the T cells, and expanding the numbers of selected T cells may be carried out as described herein with respect to other aspects of the disclosure.

In certain embodiments, this disclosure relates to a method comprising administering to the mammal nonmyeloablative lymphodepleting chemotherapy. The nonmyeloablative lymphodepleting chemotherapy can be any suitable such therapy, which can be administered by any suitable route. The nonmyeloablative lymphodepleting chemotherapy can comprise the administration of cyclophosphamide and fludarabine, particularly if the cancer is an HPV-positive cancer, which can be metastatic. A preferred route of administering cyclophosphamide and fludarabine is intravenously. Likewise, any suitable dose of cyclophosphamide and fludarabine can be administered. Preferably, around 60 mg/kg of cyclophosphamide is administered for two days after which around 25 mg/m² fludarabine is administered for five days, particularly if the cancer is an HPV-positive cancer. In an embodiment, the nonmyeloablative lymphodepleting chemotherapy is administered prior to administering the T cells.

In certain embodiments, this disclosure relates to a method comprising, after administering the nonmyeloablative lymphodepleting chemotherapy, administering to the mammal the population of HPV-specific T cells prepared by any of the methods described herein.

The T-cells can be administered by any suitable route as known in the art. Preferably, the T-cells are administered as an intra-arterial or intravenous infusion, which preferably lasts about 30 to about 60 minutes. Other examples of routes of administration include intraperitoneal, intrathecal and intralymphatic.

In certain embodiments, methods described herein may further comprise combining the population of HPV-specific T cells with a pharmaceutically acceptable carrier. The pharmaceutically acceptable carrier may be any pharmaceutically acceptable carrier that is suitable for adoptive cell therapy. For example, the pharmaceutically acceptable carrier may include any isotonic carrier such as, for example, normal saline (about 0.90% w/v of NaCl in water, about 300 mOsm/L NaCl in water, or about 9.0 g NaCl per liter of water), about 5% dextrose in water, or Ringer's lactate. In an embodiment, the pharmaceutically acceptable carrier is supplemented with human serum albumen.

In certain embodiments, this disclosure relates to a method comprising enriching cultured T cells for CD8⁺ T cells prior to rapid expansion of the cells. Following culture of the T cells, the T cells are depleted of CD4⁺ cells and enriched for CD8⁺ cells using, for example, a CD8 microbead separation. Without being bound to a particular theory, it is believed that CD8⁺ enrichment of some T cell cultures reveals in vitro tumor recognition that may not be evident in the bulk culture and improved in vitro recognition of tumor in other cultures. Additionally, the enriched CD8⁺ T cells are believed to behave more reliably and predictably in clinical scale rapid expansions than the bulk T cells.

In certain embodiments, this disclosure relates to a method comprising enriching cultured T cells for CD⁺ T cells prior to rapid expansion of the cells. Following culture of the T cells, the T cells are depleted of CD8⁺ cells and enriched for CD4⁺ cells using, for example, a CD4 microbead separation.

Expansion of the numbers of T cells can be accomplished by any of a number of methods as are known in the art as described in, for example, U.S. Pat. Nos. 8,034,334; 8,383,099; U.S. Patent Application Publication No. 2012/0244133; Dudley et al., J. Immunother., 26:332-42 (2003); and Riddell et al., J. Immunol. Methods, 128:189-201 (1990). For example, the numbers of T cells can be rapidly expanded using non-specific T-cell receptor stimulation in the presence of feeder lymphocytes and either interleukin-2 (IL-2) or interleukin-15 (IL-15), with IL-2 being preferred. The non-specific T-cell receptor stimulus can include around 30 ng/mL of OKT3, a mouse monoclonal anti-CD3 antibody (available from Ortho-McNeil, Raritan, N.J.). Alternatively, the number of T cells can be rapidly expanded by stimulation in vitro with an antigen (one or more, including antigenic portions thereof, such as epitope(s), or a cell) of the cancer, which can be optionally expressed from a vector in the presence of a T-cell growth factor, such as 300 IU/mL IL-2 or IL-15, with IL-2 being preferred. The numbers of in vitro-induced T-cells may be rapidly expanded by re-stimulation with the same antigen(s) of the cancer pulsed onto antigen-presenting cells.

In an embodiment, a T-cell growth factor that promotes the growth and activation of the autologous T cells is administered to the mammal either concomitantly with the autologous T cells or subsequently to the autologous T cells. The T-cell growth factor can be any suitable growth factor that promotes the growth and activation of the autologous T-cells. Examples of suitable T-cell growth factors include interleukin (IL)-2, IL-7, IL-15, and IL-12, which can be used alone or in various combinations, such as IL-2 and IL-7, IL-2 and IL-15, IL-7 and IL-15, IL-2, IL-7 and IL-15, IL-12 and IL-7, IL-12 and IL-15, or IL-12 and IL2. IL-12 is a preferred T-cell growth factor.

In an embodiment, the autologous T-cells are modified to express a T-cell growth factor that promotes the growth and activation of the autologous T-cells. The T-cell growth factor can be administered by any suitable route. If more than one T-cell growth factor is administered, they can be administered simultaneously or sequentially, in any order, and by the same route or different routes. Preferably, the T-cell growth factor, such as IL-2, is administered intravenously as a bolus injection. The dosage of the T-cell growth factor may be chosen based on patient tolerance. For example, the T-cell growth factor may be administered until one or more limiting adverse events occur. Desirably, the dosage of the T-cell growth factor, such as IL-2, is what is considered by those of ordinary skill in the art to be high. Preferably, a dose of about 720,000 IU/kg of IL-2 is administered three times daily until tolerance, particularly when the cancer is an HPV-positive cancer. Preferably, about 5 to about 15 doses of IL-2 are administered, with an average of around 9 doses.

In an embodiment, the autologous T-cells may be modified to express a T cell receptor (TCR) having specificity for an HPV epitope disclosed herein. Suitable methods of modification are known in the art. See, for instance, Green and Sambrook and Ausubel, supra. For example, the T cells may be transduced to express a T cell receptor (TCR) having antigenic specificity for an HPV antigen using transduction techniques described in Heemskerk et al. Hum Gene Ther. 19:496-510 (2008) and Johnson et al. Blood 114:535-46 (2009).

The TCR materials can be modified in any number of ways, such that the therapeutic or prophylactic efficacy of the TCR materials is increased through the modification. For instance, the TCR materials can be conjugated either directly or indirectly through a bridge to a targeting moiety. The practice of conjugating compounds, e.g., TCR materials, to targeting moieties is known in the art. See, for instance, Wadwa et al., J. Drug Targeting 3:111 (1995) and U.S. Pat. No. 5,087,616. The term “targeting moiety” as used herein, refers to any molecule or agent that specifically recognizes and binds to a cell-surface receptor, such that the targeting moiety directs the delivery of the TCR materials to a population of cells on which surface the receptor is expressed. Targeting moieties include, but are not limited to, antibodies, or fragments thereof, peptides, hormones, growth factors, cytokines, and any other natural or non-natural ligands, which bind to cell surface receptors (e.g., Epithelial Growth Factor Receptor (EGFR), T cell receptor (TCR), B-cell receptor (BCR), CD28, Platelet-derived Growth Factor Receptor (PDGF), nicotinic acetylcholine receptor (nAChR), etc.). The term “bridge” as used herein, refers to any agent or molecule that links the TCR materials to the targeting moiety. One of ordinary skill in the art recognizes that sites on the TCR materials, which are not necessary for the function of the TCR materials, are ideal sites for attaching a bridge and/or a targeting moiety, provided that the bridge and/or targeting moiety, once attached to the TCR materials, do(es) not interfere with the function of the TCR materials, i.e., the ability to bind to HPV 16 E6; or to detect, treat, or prevent cancer, HPV 16 infection, or HPV-positive premalignancy.

It is contemplated that the pharmaceutical compositions, TCRs (including functional variants thereof), polypeptides, proteins, nucleic acids, recombinant expression vectors, particles, host cells, or populations of cells can be used in methods of treating or preventing cancer, HPV 16 infection, or HPV-positive premalignancy. Without being bound to a particular theory, the TCRs (and functional variants thereof) are believed to bind specifically to HPV 16 protein, such that the TCR (or related polypeptide or protein and functional variants thereof), when expressed by a cell, is able to mediate an immune response against a target cell expressing HPV 16 protein. In this regard, this disclosure relates to a method of treating or preventing a condition in a mammal, comprising administering to the mammal any of the pharmaceutical compositions, TCRs (and functional variants thereof), polypeptides, or proteins described herein, any nucleic acid or recombinant expression vector comprising a nucleotide sequence encoding any of the TCRs (and functional variants thereof), polypeptides, proteins described herein, or any host cell or population of cells comprising a recombinant vector which encodes any of the TCRs (and functional variants thereof), polypeptides, or proteins described herein, in an amount effective to treat or prevent the condition in the mammal, wherein the condition is cancer, HPV 16 infection, or HPV-positive premalignancy.

The terms “treat,” and “prevent” as well as words stemming therefrom, as used herein, do not necessarily imply 100% or complete treatment or prevention. Rather, there are varying degrees of treatment or prevention of which one of ordinary skill in the art recognizes as having a potential benefit or therapeutic effect. In this respect, the methods can provide any amount of any level of treatment or prevention of a condition in a mammal. Furthermore, the treatment or prevention provided by the method can include treatment or prevention of one or more conditions or symptoms of the condition, e.g., cancer, being treated or prevented. For example, treatment or prevention can include promoting the regression of a tumor. Also, for purposes herein, “prevention” can encompass delaying the onset of the condition, or a symptom or condition thereof.

Also provided is a method of detecting the presence of a condition in a mammal. The method comprises contacting a sample comprising one or more cells from the mammal with any of the TCRs (and functional variants thereof), polypeptides, proteins, nucleic acids, recombinant expression vectors, host cells, populations of cells, antibodies, or antigen binding portions thereof, or pharmaceutical compositions described herein, thereby forming a complex, and detecting the complex, wherein detection of the complex is indicative of the presence of the condition in the mammal, wherein the condition is cancer, HPV 16 infection, or HPV-positive premalignancy.

With respect to the method of detecting a condition in a mammal, the sample of cells can be a sample comprising whole cells, lysates thereof, or a fraction of the whole cell lysates, e.g., a nuclear or cytoplasmic fraction, a whole protein fraction, or a nucleic acid fraction.

For purposes of the detecting method, the contacting can take place in vitro or in vivo with respect to the mammal. Preferably, the contacting is in vitro. Also, detection of the complex can occur through any number of ways known in the art. For instance, the TCRs (and functional variants thereof), polypeptides, proteins, nucleic acids, recombinant expression vectors, host cells, populations of cells, or antibodies, or antigen binding portions thereof, described herein, can be labeled with a detectable label such as, for instance, a radioisotope, a fluorophore (e.g., fluorescein isothiocyanate (FITC), phycoerythrin (PE)), an enzyme (e.g., alkaline phosphatase, horseradish peroxidase), and element particles (e.g., gold particles).

For purposes of the methods, wherein host cells or populations of cells are administered, the cells can be cells that are allogeneic or autologous to the mammal. Preferably, the cells are autologous to the mammal.

With respect to the methods, the cancer can be any cancer, including any of acute lymphocytic cancer, acute myeloid leukemia, alveolar rhabdomyosarcoma, bone cancer, brain cancer, breast cancer, cancer of the anus, anal canal, or anorectum, cancer of the eye, cancer of the intrahepatic bile duct, cancer of the joints, cancer of the neck, gallbladder, or pleura, cancer of the nose, nasal cavity, or middle ear, cancer of the oral cavity, cancer of the vagina, cancer of the vulva, chronic lymphocytic leukemia, chronic myeloid cancer, colon cancer, esophageal cancer, cervical cancer, gastrointestinal carcinoid tumor, glioma, Hodgkin lymphoma, hypopharynx cancer, kidney cancer, larynx cancer, liver cancer, lung cancer, malignant mesothelioma, melanoma, multiple myeloma, nasopharynx cancer, non-Hodgkin lymphoma, cancer of the oropharynx, ovarian cancer, cancer of the penis, pancreatic cancer, peritoneum, omentum, and mesentery cancer, pharynx cancer, prostate cancer, rectal cancer, renal cancer, skin cancer, small intestine cancer, soft tissue cancer, stomach cancer, testicular cancer, thyroid cancer, cancer of the uterus, ureter cancer, and urinary bladder cancer. A preferred cancer is cancer is cancer of the uterine cervix, oropharynx, anus, anal canal, anorectum, vagina, vulva, or penis. A particularly preferred cancer is HPV 16-positive cancer. While the cancers most commonly associated with HPV 16 infection include cancer of the uterine cervix, oropharynx, anus, anal canal, anorectum, vagina, vulva, and penis, the methods may be used to treat any HPV 16-positive cancer, including those that occur at other anatomical areas.

In certain embodiments, this disclosure contemplates using TCRs as targeting system. In certain embodiments, TCR-functionalize nanoparticles, membrane vesicles, can contain within or on the surface an anticancer agent or other payload or toxic agent for delivery to the tumor. In certain embodiments, the TCRs or specific binding polypeptides can be conjugated to micron sized particles, or nanoparticles such as metal, iron oxide nanoparticles with a core diameter of 100-5 nm, or polymeric polymers. Such particles can be further conjugated to anticancer agents.

In certain embodiments, the anti-cancer agent for use in a combination therapy or for conjugation to vehicles of a targeting system may be an anticancer agent selected from abemaciclib, abiraterone acetate, methotrexate, paclitaxel, adriamycin, acalabrutinib, brentuximab vedotin, ado-trastuzumab emtansine, aflibercept, afatinib, netupitant, palonosetron, imiquimod, aldesleukin, alectinib, alemtuzumab, pemetrexed disodium, copanlisib, melphalan, brigatinib, chlorambucil, amifostine, aminolevulinic acid, anastrozole, apalutamide, aprepitant, pamidronate disodium, exemestane, nelarabine, arsenic trioxide, ofatumumab, atezolizumab, bevacizumab, avelumab, axicabtagene ciloleucel, axitinib, azacitidine, carmustine, belinostat, bendamustine, inotuzumab ozogamicin, bevacizumab, bexarotene, bicalutamide, bleomycin, blinatumomab, bortezomib, bosutinib, brentuximab vedotin, brigatinib, busulfan, irinotecan, capecitabine, fluorouracil, carboplatin, carfilzomib, ceritinib, daunorubicin, cetuximab, cisplatin, cladribine, cyclophosphamide, clofarabine, cobimetinib, cabozantinib-S-malate, dactinomycin, crizotinib, ifosfamide, ramucirumab, cytarabine, dabrafenib, dacarbazine, decitabine, daratumumab, dasatinib, defibrotide, degarelix, denileukin diftitox, denosumab, dexamethasone, dexrazoxane, dinutuximab, docetaxel, doxorubicin, durvalumab, rasburicase, epirubicin, elotuzumab, oxaliplatin, eltrombopag olamine, enasidenib, enzalutamide, eribulin, vismodegib, erlotinib, etoposide, everolimus, raloxifene, toremifene, panobinostat, fulvestrant, letrozole, filgrastim, fludarabine, flutamide, pralatrexate, obinutuzumab, gefitinib, gemcitabine, gemtuzumab ozogamicin, glucarpidase, goserelin, propranolol, trastuzumab, topotecan, palbociclib, ibritumomab tiuxetan, ibrutinib, ponatinib, idarubicin, idelalisib, imatinib, talimogene laherparepvec, ipilimumab, romidepsin, ixabepilone, ixazomib, ruxolitinib, cabazitaxel, palifermin, pembrolizumab, ribociclib, tisagenlecleucel, lanreotide, lapatinib, olaratumab, lenalidomide, lenvatinib, leucovorin, leuprolide, lomustine, trifluridine, olaparib, vincristine, procarbazine, mechlorethamine, megestrol, trametinib, temozolomide, methylnaltrexone bromide, midostaurin, mitomycin C, mitoxantrone, plerixafor, vinorelbine, necitumumab, neratinib, sorafenib, nilutamide, nilotinib, niraparib, nivolumab, tamoxifen, romiplostim, sonidegib, omacetaxine, pegaspargase, ondansetron, osimertinib, panitumumab, pazopanib, interferon alfa-2b, pertuzumab, pomalidomide, mercaptopurine, regorafenib, rituximab, rolapitant, rucaparib, siltuximab, sunitinib, thioguanine, temsirolimus, thalidomide, thiotepa, trabectedin, valrubicin, vandetanib, vinblastine, vemurafenib, vorinostat, zoledronic acid, or combinations thereof.

In certain embodiments, the chemotherapy agent is an anti-PD-1, anti-PD-L1 anti-CTLA4 antibody or combinations thereof, such as an anti-CTLA4 (e.g., ipilimumab, tremelimumab) and anti-PD1 (e.g., nivolumab, pembrolizumab, cemiplimab) and anti-PD-L1 (e.g., atezolizumab, avelumab, durvalumab).

In certain embodiments, administration is in a subject with a lymphodepleted environment due to prior or concurrent administration of lymphodepleting agents. In certain embodiments, lymphodepleting agents (e.g., cyclophosphamide and fludarabine).

In certain embodiments, this disclosure relates to compositions comprising a polypeptide comprising or consisting of epitopes selected from of SEQ ID NOs: 1-9. In certain embodiments, the polypeptide consists of an N-terminus having the amino acid sequence of SEQ ID NO: 1-9. In certain embodiments, the polypeptide consists of a C-terminus having the amino acid sequence of SEQ ID NO: 1-9. In certain embodiments, the polypeptide is conjugated to a label.

In certain embodiments, this disclosure relates to multimeric complex comprising a polypeptide disclosed herein. In certain embodiments, the polypeptide is specifically binding an MHC-I protein.

In certain embodiments, this disclosure relates to methods comprising: contacting a polypeptide or multimeric complex of a polypeptide of SEQ ID NOs: 1-9 with a sample from a subject at risk of, exhibiting symptoms of, or diagnosed with cancer comprising T cells with receptors that specifically bind the polypeptide providing polypeptide bound T cells; and isolating the polypeptide bound T cells providing isolated T cells that bind to the polypeptide.

In certain embodiments, the sample comprises tumor-infiltrating lymphocytes isolated from primary tumors or metastatic lymph nodes from the subject. In certain embodiments, the isolated T cells are CD8+.

### EXAMPLES

### HPV-Specific CD8 T Cells in Head and Neck Cancer

The differentiation states of HPV-specific CD8 T cell responses in the tumor microenvironment (TME) of HPV-positive head and neck squamous cell carcinoma (HNSCC) were examined patients. Several HPV-specific CD8 T cell epitopes were identified in these HNSCC patients. A detailed analysis of HPV-specific CD8 T cell responses in the tumor were performed. HPV-specific CD8 T cell epitopes derived from HPV E2, E5 and E6 were identified. These epitopes were analyzed in tumor-infiltrating HPV-specific CD8 T cells using MHC-I tetramers. HPV-specific CD8 T cells uniformly expressed high levels of PD-1 and were readily detectable in primary tumors and metastatic lymph nodes, ranging from 0.1% to 10% of tumor-infiltrating CD8 T cells for a given epitope, but below the limit of detection (<0.02%) in the peripheral blood. Single-cell RNA-seq analyses of tumor-infiltrating HPV-specific PD-1+CD8 T cells revealed three transcriptionally distinct subsets. The first subset expressed high levels of the canonical transcription factor TCF7 and other genes associated with PD-1+ stem-like resource CD8 T cells that are associated with maintaining T cell responses. The second subset expressed the highest levels of effector molecules such as IFNG and was the most activated (TCR signals) consistent with it representing a transitory cell population. The third subset was characterized by a terminally exhausted gene signature including high expression of various inhibitory receptors.

TCR analysis of the HPV-specific CD8 T cells showed that cells of the same TCR clonotype were present in all three differentiation states, suggesting a lineage relationship in which PD-1+TCF-1+ stem-like resource cells give rise to the transitory and exhausted CD8 T cells in the tumor microenvironment. Pseudotime analyses further supported this lineage relationship with resource cells giving rise to more differentiated progeny. In line with these observations, in vitro proliferation experiments demonstrated that, in contrast to terminally differentiated cells, HPV-specific stem-like resource cells possess high proliferative potential and, upon antigenic stimulation, undergo several rounds of proliferation and acquire a more differentiated cell state characterized by the expression of effector molecules such as granzyme B. Taken together, the presence of HPV-specific PD-1+TCF-1+ stem-like CD8 T cells with high proliferative capacity shows that the cellular machinery to respond to PD-1 blockade exists in HNSCC and supports that the combination of PD-1 therapy and therapeutic vaccination could be an effective treatment in this malignancy.

### Identification of HPV-Specific CD8 T Cells in TILs of HNSCC Patients

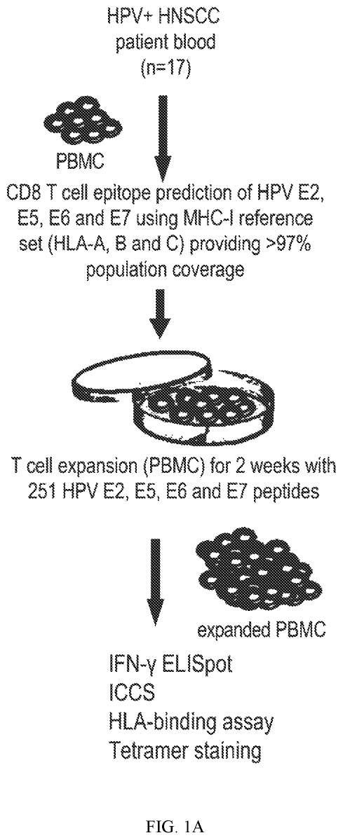

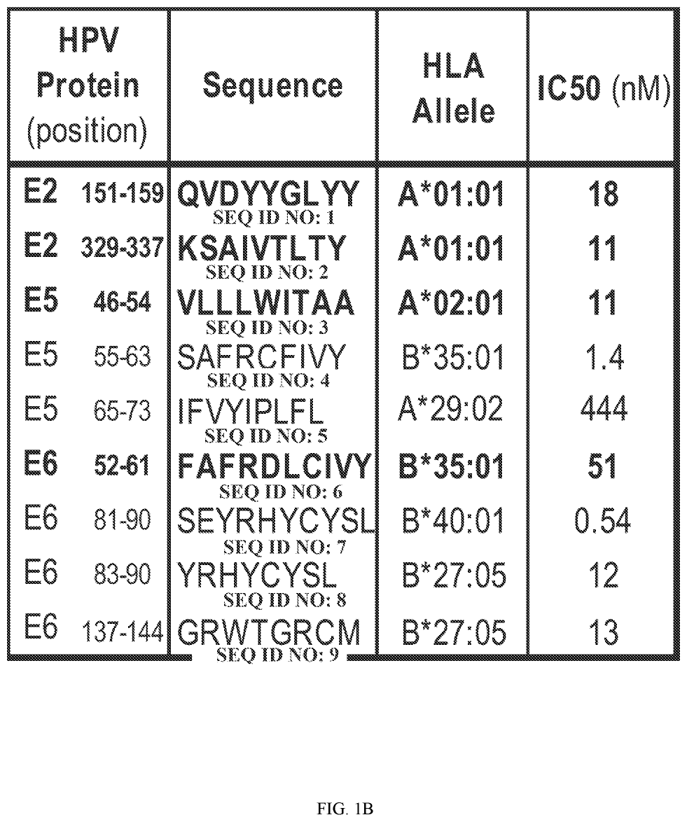

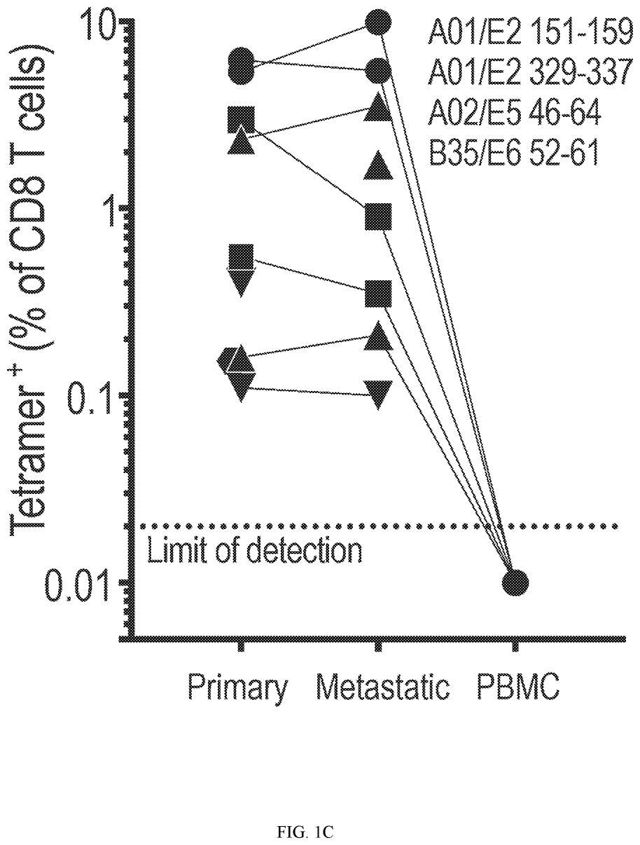

HPV offers a unique opportunity to study tumor-reactive CD8 T cells against a set of defined virus-derived tumor-associated antigens. HPV-specific CD8 T cell epitopes were identified with the goal of characterizing HPV-specific tumor-reactive CD8 T cells using MHC class I tetramers. CD8 T cell epitopes, presented by 27 HLA class I alleles providing a population coverage of >97%, were predicted using the Immune Epitope Database (FIG. 1A). To identify patient-specific HPV epitopes, PBMC were isolated from patients and T cells were expanded in the presence of 251 predicted HPV peptides (percentile rank ≤1). These antigen-expanded cells were tested for reactivity against small peptide pools or single peptides using ELISpot, intra-cellular cytokine staining (ICCS), and tetramer staining. Overall, 43 different peptides reacted in the interferon-y ELISpot assay (22 HPV-E2 peptides, 10 HPV-E5 peptides, 9 HPV-E6 peptides and 2 HPV-E7 peptides) with several peptides being recognized by multiple patients. Reactive peptides were confirmed by in vitro stimulation and intra-cellular cytokine staining (ICS) of expanded T cells. Patients were HLA-typed and peptide-HLA-allele pairs were predicted in silico. In vitro HLA-binding assays were performed to determine the peptide: MHC-I binding affinity prior to the generation and testing of MHC-I tetramers.

Using the above analysis methodology, nine CD8 T cell epitopes were identified (FIG. 1B). Seven different MHC-I tetramers were generated with epitopes derived from HPV E2, E5 and E6. HPV-specific tumor-reactive CD8 T cells were detected directly ex vivo in TILs isolated from primary tumors and metastatic lymph nodes. The frequency of HPV-specific CD8 T cells varied widely between patients and epitopes ranging from 0.1% to 10% of tumor-infiltrating CD8 T cells. All tetramer-positive cells expressed high levels of PD-1. HPV-specific CD8 T cells to a given epitope were present at similar frequencies in both primary tumor and metastatic lymph node in the same individual (FIG. 1C). In comparison to the tumor, tetramer-positive cells in patient-matched peripheral blood directly could not be detected directly by ex vivo staining, suggesting very low numbers of HPV-specific CD8 T cells (<0.02% of CD8 T cells) in circulation. These results indicate extremely low frequencies of tumor-specific CD8 T cells in peripheral blood. HPV-specific CD8 T cells in HPV-positive HNSCC and neo-antigen-specific CD8 T cells in ovarian cancer only being detectable after ex vivo expansion. Furthermore, CD8 T cell clones proliferating in response to PD-1 blockade are close to or below the limit of detection in the peripheral blood prior to treatment initiation.

The uniformly high expression of PD-1 among tetramer-positive (E2, E5, and E6) CD8 T cells in the tumor indicate that E2, E5 and E6 are expressed and the respective epitopes are being presented within the TME, resulting in persistent TCR engagement of HPV-specific CD8 T cells. Together, these data indicate that HPV-specific CD8 T cells can account for a substantial proportion of tumor-infiltrating CD8 T cells, and that while cells of the same antigen specificity can be expanded from PBMC of patients, their frequency in the blood is quite low under steady state conditions.

### HPV positive T Cell Expansion

PBMCs from peripheral blood of patients with HPV+ head and neck tumors were used for in vitro T cell expansion. Potential CD8 T cell epitopes derived from HPV proteins E2, E5, E6 and E7, and presented by human leukocyte antigens (HLA-A, B and C) covering 97% of the population were predicted using the Immune Epitope Database (IEDB) prediction algorithm. Predicted peptides were synthesized and ultimately resuspended in DMSO. A peptide pool was prepared containing 250 different 8-11 amino acid-long peptides from proteins E2 (124 peptides), E5 (55 peptides), E6 (50 peptides), E7 (21 peptides). PBMCs were cultured in complete CTS OpTmizer™M medium (CTS supplement, substituted with L-Glutamine, Penicillin/Streptomycin and 2% human serum) in presence of the HPV-peptide pool (1 μg/ml per peptide), rIL-2 (50IU/ml), rIL-7 (25 ng/ml) and rIL-15 (25 ng/ml). HPV-peptides were only added at day 1 of culture, whereas cytokines were supplemented whenever cells were split during the 2-weeks expansion period. At day 13 of cell culture, expanded PBMCs were washed and rested overnight in cytokine-free media.

### Identification of Epitopes by ELISPOT and Intracellular Cytokine Staining (ICS)

Expanded and rested PBMCs were plated in an INFgamma-ELISPOT, stimulated overnight with individual HPV peptides and positive peptides were identified. For each recognized peptide sequence, the binding affinity to the responder's HLA alleles was predicted in silico (IEBD). The HLA allele with the highest binding affinity for each epitope was retained as HLA-peptide pair and confirmed by ICS.

### Generation of HPV-Specific Tetramers

HLA-peptide pairs were identified and the binding affinity of the peptides to the respective HLA alleles was determined by in vitro binding assays. HLA-tetramers were generated. Tetramer staining was tested on expanded PBMCs and frequencies of tetramer-positive CD8 T cells were similar to frequencies of IFNy-positive CD8 T cells previously quantified by ICS. The following HLA-tetramers: HLA-A*01:01-E2329-337 (peptide sequence KSAIVTLTY), HLA-A*01:01-E2 151-159 (peptide sequence QVDYYGLYY) and HLA-A*02:01-E546-54 (peptide sequence VLLLWITAA).

### Sorting of HPV-Positive CD8 T Cells from Lymphocytes Infiltrating Tumor and Metastasized LN

HPV-positive head and neck cancer patients possessing HLA-A*01:01 or HLA-A*02:01 alleles were identified. Tumor-infiltrating lymphocytes (TILs) were isolated from their tumors and metastasized lymph node tissues and cryopreserved. At the day of sorting, cells were thawed and stained with one of the 3 above described tetramers. CD3posCD19negCD14negCD16negCD4neg CD8pos double-tetramer positive cells were sorted into PBS with 2% FBS and single cell RNA sequencing performed using 10x Genomics Chromium Single Cell Immune Profiling Solution using Cell Ranger. Cells with a single TCR alpha and beta chain pair were selected for further analysis.

What is claimed is:

1. A T cell receptor (TCR) having specificity for human papillomavirus (HPV) 16 E2 or E5 and comprising a human variable region and a constant region.

2. The TCR of claim 1, wherein the TCR has specificity for HPV 16 E2 151-159 comprising the amino acid sequence of SEQ ID NO: 1.

3. The TCR of claim 1, wherein the TCR has specificity for HPV 16 E2 329-337 comprising the amino acid sequence of SEQ ID NO: 2.

4. The TCR of claim 1, wherein the TCR has specificity for HPV 16 E5 46-54 comprising the amino acid sequence of SEQ ID NO: 3.

5. The TCR of claim 1, comprising the amino acid sequences of the alpha chain and/or beta chain or comprising CDRs 1, 2, and 3 therein, or variants thereof.

6. A TCR of claim 1 with a heterologous segment or a variant with one amino acid mutation, addition, and/or deletion such that the TCR is not naturally occurring.

7. A polypeptide having specificity for human papillomavirus (HPV) 16 E2 or E5 and comprising a human variable region.

8. The polypeptide of claim 7 having specificity for HPV 16 E2 151-159 comprising the amino acid sequence of SEQ ID NO: 1.

9. The polypeptide of claim 7 having specificity for HPV 16 E2 329-337 comprising the amino acid sequence of SEQ ID NO: 2.

10. The polypeptide of claim 7 having specificity for HPV 16 E5 46-54 comprising the amino acid sequence of SEQ ID NO: 3.

11. The polypeptide of claim 7, comprising the amino acid sequences of the alpha chain and/or beta chain or comprising CDRs 1, 2, and/or 3 therein.

12. The polypeptide of claim 7, comprising a heterologous peptide sequence, fluorescent tag, or other label.

13-17. (canceled)

18. A heterologous nucleic acid comprising a nucleotide sequence encoding the TCR of claim 1.

19. A recombinant expression vector comprising the nucleic acid of claim 18.

20. An isolated host cell or population of cells comprising a recombinant expression vector of claim 19.

21-35. (canceled)


SEQUENCE LISTING (197 sequences)

SEQ ID NO: 1 - protein, 9 aa, synthetic construct
  REGION 1..9: note = Synthetic construct
QVDYYGLYY

SEQ ID NO: 2 - protein, 9 aa, synthetic construct
  REGION 1..9: note = Synthetic construct
KSAIVTLTY

SEQ ID NO: 3 - protein, 9 aa, synthetic construct
  REGION 1..9: note = Synthetic construct
VLLLWITAA

SEQ ID NO: 4 - protein, 9 aa, synthetic construct
  REGION 1..9: note = Synthetic construct
SAFRCFIVY

SEQ ID NO: 5 - protein, 9 aa, synthetic construct
  REGION 1..9: note = Synthetic construct
IFVYIPLFL

SEQ ID NO: 6 - protein, 10 aa, synthetic construct
  REGION 1..10: note = Synthetic construct
FAFRDLCIVY

SEQ ID NO: 7 - protein, 9 aa, synthetic construct
  REGION 1..9: note = Synthetic construct
SEYRHYCYS

SEQ ID NO: 8 - protein, 8 aa, synthetic construct
  REGION 1..8: note = Synthetic construct
YRHYCYSL

SEQ ID NO: 9 - protein, 8 aa, synthetic construct
  REGION 1..8: note = Synthetic construct
GRWTGRCM

SEQ ID NO: 10 - protein, 118 aa, synthetic construct
  REGION 1..118: note = Synthetic construct
AQTVTQSQPEMSVQEAETVTLSCTYDTSESDYYLFWYKQPPSRQMILVIRQEAYKQQNATENRFSVNFQKAAKSFSLKISDSQLGDAAMYFCAYNCPEPSDSSYKLIFGSGTRLLVRP

SEQ ID NO: 11 - protein, 112 aa, synthetic construct
  REGION 1..112: note = Synthetic construct
DGGITQSPKYLFRKEGQNVTLSCEQNLNHDAMYWYRQDPGQGLRLIYYSQIVNDFQKGDIAEGYSVSREKKESFPLTVTSAQKNPTAFYLCASSMLLNQPQHFGDGTRLSIL

SEQ ID NO: 12 - protein, 117 aa, synthetic construct
  REGION 1..117: note = Synthetic construct
KNQVEQSPQSLIILEGKNCTLQCNYTVSPFSNLRWYKQDTGRGPVSLTIMTFSENTKSNGRYTATLDADTKQSSLHITASQLSDSASYICVVSAVEDSGGGADGLTFGKGTHLIIQP

SEQ ID NO: 13 - protein, 114 aa, synthetic construct
  REGION 1..114: note = Synthetic construct
DGGITQSPKYLFRKEGQNVTLSCEQNLNHDAMYWYRQDPGQGLRLIYYSQIVNDFQKGDIAEGYSVSREKKESFPLTVTSAQKNPTAFYLCASSPILQGQYGYTFGSGTRLTVV

SEQ ID NO: 14 - protein, 114 aa, synthetic construct
  REGION 1..114: note = Synthetic construct
AQTVTQSQPEMSVQEAETVTLSCTYDTSESDYYLFWYKQPPSRQMILVIRQEAYKQQNATENRFSVNFQKAAKSFSLKISDSQLGDAAMYFCAYRSAGAQKLVFGQGTRLTINP

SEQ ID NO: 15 - protein, 115 aa, synthetic construct
  REGION 1..115: note = Synthetic construct
DTEVTQTPKHLVMGMTNKKSLKCEQHMGHRAMYWYKQKAKKPPELMFVYSYEKLSINESVPSRFSPECPNSSLLNLHLHALQPEDSALYLCASSLEGRDLIFEQFFGPGTRLTVL

SEQ ID NO: 16 - protein, 115 aa, synthetic construct
  REGION 1..115: note = Synthetic construct
GQQLNQSPQSMFIQEGEDVSMNCTSSSIFNTWLWYKQDPGEGPVLLIALYKAGELTSNGRLTAQFGITRKDSFLNISASIPSDVGIYFCAGQRLETGGGADGLTFGKGTHLIIQP

SEQ ID NO: 17 - protein, 113 aa, synthetic construct
  REGION 1..113: note = Synthetic construct
DVKVTQSSRYLVKRTGEKVFLECVQDMDHENMFWYRQDPGLGLRLIYFSYDVKMKEKGDIPEGYSVSREKKERFSLILESASTNQTSMYLCASSSYRDNQPQHFGDGTRLSIL

SEQ ID NO: 18 - protein, 112 aa, synthetic construct
  REGION 1..112: note = Synthetic construct
QKEVEQNSGPLSVPEGAIASLNCTYSDRGSQSFFWYRQYSGKSPELIMSIYSNGDQEDGRFTAQLNKASQYVSLLIRDSQPSDSATYLCAVYPSGGYQKVTFGIGTKLQVIP

SEQ ID NO: 19 - protein, 115 aa, synthetic construct
  REGION 1..115: note = Synthetic construct
DTEVTQTPKHLVMGMTNKKSLKCEQHMGHRAMYWYKQKAKKPPELMFVYSYEKLSINESVPSRFSPECPNSSLLNLHLHALQPEDSALYLCASSQDDSGAGNTIYFGEGSWLTVV

SEQ ID NO: 20 - protein, 114 aa, synthetic construct
  REGION 1..114: note = Synthetic construct
AQTVTQSQPEMSVQEAETVTLSCTYDTSENNYYLFWYKQPPSRQMILVIRQEAYKQQNATENRFSVNFQKAAKSFSLKISDSQLGDTAMYFCASPGAGSYQLTFGKGTKLSVIP

SEQ ID NO: 21 - protein, 114 aa, synthetic construct
  REGION 1..114: note = Synthetic construct
DVKVTQSSRYLVKRTGEKVFLECVQDMDHENMFWYRQDPGLGLRLIYFSYDVKMKEKGDIPEGYSVSREKKERFSLILESASTNQTSMYLCASSPRVASVETQYFGPGTRLLVL

SEQ ID NO: 22 - protein, 115 aa, synthetic construct
  REGION 1..115: note = Synthetic construct
AQKVTQAQTEISVVEKEDVTLDCVYETRDTTYYLFWYKQPPSGELVFLIRRNSFDEQNEISGRYSWNFQKSTSSFNFTITASQVVDSAVYFCALSPEGFGNVLHCGSGTQVIVLP

SEQ ID NO: 23 - protein, 115 aa, synthetic construct
  REGION 1..115: note = Synthetic construct
DTAVSQTPKYLVTQMGNDKSIKCEQNLGHDTMYWYKQDSKKFLKIMFSYNNKELIINETVPNRFSPKSPDKAHLNLHINSLELGDSAVYFCASSQGASASYNEQFFGPGTRLTVL

SEQ ID NO: 24 - protein, 110 aa, synthetic construct
  REGION 1..110: note = Synthetic construct
KNEVEQSPQNLTAQEGEFITINCSYSVGISALHWLQQHPGGGIVSLFMLSSGKKKHGRLIATINIQEKHSSLHITASHPRDSAVYICAVHSGGGADGLTFGKGTHLIIQP

SEQ ID NO: 25 - protein, 113 aa, synthetic construct
  REGION 1..113: note = Synthetic construct
DGGITQSPKYLFRKEGQNVTLSCEQNLNHDAMYWYRQDPGQGLRLIYYSQIVNDFQKGDIAEGYSVSREKKESFPLTVTSAQKNPTAFYLCASSMMGQIDVLTFGAGSRLTVL

SEQ ID NO: 26 - protein, 115 aa, synthetic construct
  REGION 1..115: note = Synthetic construct
DAGVIQSPRHEVTEMGQEVTLRCKPISGHDYLFWYRQTMMRGLELLIYFNNNVPIDDSGMPEDRFSAKMPNASFSTLKIQPSEPRDSAVYFCASSTPRDLNTEAFFGQGTRLTVV

SEQ ID NO: 27 - protein, 115 aa, synthetic construct
  REGION 1..115: note = Synthetic construct
DTEVTQTPKHLVMGMTNKKSLKCEQHMGHRAMYWYKQKAKKPPELMFVYSYEKLSINESVPSRFSPECPNSSLLNLHLHALQPEDSALYLCASSQEGLDTSYEQYFGPGTRLTVT

SEQ ID NO: 28 - protein, 111 aa, synthetic construct
  REGION 1..111: note = Synthetic construct
KQEVTQIPAALSVPEGENLVLNCSFTDSAIYNLQWFRQDPGKGLTSLLLIQSSQREQTSGRLNASLDKSSGRSTLYIAASQPGDSATYLCAVDTGGFKTIFGAGTRLFVKA

SEQ ID NO: 29 - protein, 113 aa, synthetic construct
  REGION 1..113: note = Synthetic construct
EAQVTQNPRYLITVTGKKLTVTCSQNMNHEYMSWYRQDPGLGLRQIYYSMNVEVTDKGDVPEGYKVSRKEKRNFPLILESPSPNQTSLYFCASSSGDGNYGYTFGSGTRLTVV

SEQ ID NO: 30 - protein, 111 aa, synthetic construct
  REGION 1..111: note = Synthetic construct
QKEVEQNSGPLSVPEGAIASLNCTYSDRGSQSFFWYRQYSGKSPELIMSIYSNGDKEDGRFTAQLNKASQYVSLLIRDSQPSDSATYLCAVDSGATNKLIFGTGTLLAVQP

SEQ ID NO: 31 - protein, 118 aa, synthetic construct
  REGION 1..118: note = Synthetic construct
SAVISQKPSRDICQRGTSLTIQCQVDSQVTMMFWYRQQPGQSLTLIATANQGSEATYESGFVIDKFPISRPNLTFSTLTVSNMSPEDSSIYLCSVDGGVGVTSTDTQYFGPGTRLTVL

SEQ ID NO: 32 - protein, 115 aa, synthetic construct
  REGION 1..115: note = Synthetic construct
AQKVTQAQTEISVVEKEDVTLDCVYETRDTTYYLFWYKQPPSGELVFLIRRNSFDEQNEISGRYSWNFQKSTSSFNFTITASQVVDSAVYFCASEPPSNTGKLIFGQGTTLQVKP

SEQ ID NO: 33 - protein, 115 aa, synthetic construct
  REGION 1..115: note = Synthetic construct
DTEVTQTPKHLVMGMTNKKSLKCEQHMGHRAMYWYKQKAKKPPELMFVYSYEKLSINESVPSRFSPECPNSSLLNLHLHALQPEDSALYLCASSQATLRGGDTQYFGPGTRLTVL

SEQ ID NO: 34 - protein, 109 aa, synthetic construct
  REGION 1..109: note = Synthetic construct
AQRVTQPEKLLSVFKGAPVELKCNYSYSGSPELFWYVQYSRQRLQLLLRHISRESIKGFTADLNKGETSFHLKKPFAQEEDSAMYYCALAYSSASKIIFGSGTRLSIRP

SEQ ID NO: 35 - protein, 114 aa, synthetic construct
  REGION 1..114: note = Synthetic construct
EAQVTQNPRYLITVTGKKLTVTCSQNMNHEYMSWYRQDPGLGLRQIYYSMNVEVTDKGDVPEGYKVSRKEKRNFPLILESPSPNQTSLYFCASSPSLAGNEKLFFGSGTQLSVL

SEQ ID NO: 36 - protein, 112 aa, synthetic construct
  REGION 1..112: note = Synthetic construct
EAQVTQNPRYLITVTGKKLTVTCSQNMNHEYMSWYRQDPGLGLRQIYYSMNVEVTDKGDVPEGYKVSRKEKRNFPLILESPSPNQTSLYFCASGTGDRETQYFGPGTRLLVL

SEQ ID NO: 37 - protein, 116 aa, synthetic construct
  REGION 1..116: note = Synthetic construct
AQKVTQAQTEISVVEKEDVTLDCVYETRDTTYYLFWYKQPPSGELVFLIRRNSFDEQNEISGRYSWNFQKSTSSFNFTITASQVVDSAVYFCALTRATGGGNKLTFGTGTQLKVEL

SEQ ID NO: 38 - protein, 115 aa, synthetic construct
  REGION 1..115: note = Synthetic construct
GAVVSQHPSWVICKSGTSVKIECRSLDFQATTMFWYRQFPKQSLMLMATSNEGSKATYEQGVEKDKFLINHASLTLSTLTVTSAHPEDSSFYICSAQDRIGYGYTFGSGTRLTVV

SEQ ID NO: 39 - protein, 117 aa, synthetic construct
  REGION 1..117: note = Synthetic construct
AQKVTQAQTEISVVEKEDVTLDCVYETRDTTYYLFWYKQPPSGELVFLIRRNSFDEQNEISGRYSWNFQKSTSSFNFTITASQVVDSAVYFCALRKETGGGADGLTFGKGTHLIIQP

SEQ ID NO: 40 - protein, 115 aa, synthetic construct
  REGION 1..115: note = Synthetic construct
GAVVSQHPSWVICKSGTSVKIECRSLDFQATTMFWYRQFPKQSLMLMATSNEGSKATYEQGVEKDKFLINHASLTLSTLTVTSAHPEDSSFYICSAQPREGYGYTFGSGTRLTVV

SEQ ID NO: 41 - protein, 112 aa, synthetic construct
  REGION 1..112: note = Synthetic construct
AQTVTQSQPEMSVQEAETVTLSCTYDTSENNYYLFWYKQPPSRQMILVIRQEAYKQQNATENRFSVNFQKAAKSFSLKISDSQLGDTAMYFCAFDNFNKFYFGSGTKLNVKP

SEQ ID NO: 42 - protein, 117 aa, synthetic construct
  REGION 1..117: note = Synthetic construct
GAVVSQHPSWVICKSGTSVKIECRSLDFQATTMFWYRQFPKQSLMLMATSNEGSKATYEQGVEKDKFLINHASLTLSTLTVTSAHPEDSSFYICSARDGGQGTLEQYFGPGTRLTVT

SEQ ID NO: 43 - protein, 111 aa, synthetic construct
  REGION 1..111: note = Synthetic construct
KQEVTQIPAALSVPEGENLVLNCSFTDSAIYNLQWFRQDPGKGLTSLLLIQSSQREQTSGRLNASLDKSSGRSTLYIAASQPGDSATYLCAVDAGGFKTIFGAGTRLFVKA

SEQ ID NO: 44 - protein, 113 aa, synthetic construct
  REGION 1..113: note = Synthetic construct
EAQVTQNPRYLITVTGKKLTVTCSQNMNHEYMSWYRQDPGLGLRQIYYSMNVEVTDKGDVPEGYKVSRKEKRNFPLILESPSPNQTSLYFCASSLLDGNTEAFFGQGTRLTVV

SEQ ID NO: 45 - protein, 114 aa, synthetic construct
  REGION 1..114: note = Synthetic construct
DGGITQSPKYLFRKEGQNVTLSCEQNLNHDAMYWYRQDPGQGLRLIYYSQIVNDFQKGDIAEGYSVSREKKESFPLTVTSAQKNPTAFYLCASSPVAKNSYEQYFGPGTRLTVT

SEQ ID NO: 46 - protein, 113 aa, synthetic construct
  REGION 1..113: note = Synthetic construct
QQQVKQSPQSLIVQKGGISIINCAYENTAFDYFPWYQQFPGKGPALLIAIRPDVSEKKEGRFTISFNKSAKQFSLHIMDSQPGDSATYFCAARTVSGTYKYIFGTGTRLKVLA

SEQ ID NO: 47 - protein, 117 aa, synthetic construct
  REGION 1..117: note = Synthetic construct
NAGVTQTPKFQVLKTGQSMTLQCAQDMNHEYMSWYRQDPGMGLRLIHYSVGAGITDQGEVPNGYNVSRSTTEDFPLRLLSAAPSQTSVYFCASSYSPSPGLASYEQYFGPGTRLTVT

SEQ ID NO: 48 - protein, 114 aa, synthetic construct
  REGION 1..114: note = Synthetic construct
EAQVTQNPRYLITVTGKKLTVTCSQNMNHEYMSWYRQDPGLGLRQIYYSMNVEVTDKGDVPEGYKVSRKEKRNFPLILESPSPNQTSLYFCASSTTGLANEKLFFGSGTQLSVL

SEQ ID NO: 49 - protein, 111 aa, synthetic construct
  REGION 1..111: note = Synthetic construct
AQRVTQPEKLLSVFKGAPVELKCNYSYSGSPELFWYVQYSRQRLQLLLRHISRESIKGFTADLNKGETSFHLKKPFAQEEDSAMYYCALGGLTGGGNKLTFGTGTQLKVEL

SEQ ID NO: 50 - protein, 114 aa, synthetic construct
  REGION 1..114: note = Synthetic construct
EAQVTQNPRYLITVTGKKLTVTCSQNMNHEYMSWYRQDPGLGLRQIYYSMNVEVTDKGDVPEGYKVSRKEKRNFPLILESPSPNQTSLYFCASAQDLSGANVLTFGAGSRLTVL

SEQ ID NO: 51 - protein, 111 aa, synthetic construct
  REGION 1..111: note = Synthetic construct
KQEVTQIPAALSVPEGENLVLNCSFTDSAIYNLQWFRQDPGKGLTSLLLIQSSQREQTSGRLNASLDKSSGRSTLYIAASQPGDSATYLCAADTGGFKTIFGAGTRLFVKA

SEQ ID NO: 52 - protein, 113 aa, synthetic construct
  REGION 1..113: note = Synthetic construct
EAQVTQNPRYLITVTGKKLTVTCSQNMNHEYMSWYRQDPGLGLRQIYYSMNVEVTDKGDVPEGYKVSRKEKRNFPLILESPSPNQTSLYFCASTGGDYNSPLHFGNGTRLTVT

SEQ ID NO: 53 - protein, 114 aa, synthetic construct
  REGION 1..114: note = Synthetic construct
EAQVTQNPRYLITVTGKKLTVTCSQNMNHEYMSWYRQDPGLGLRQIYYSMNVEVTDKGDVPEGYKVSRKEKRNFPLILESPSPNQTSLYFCASSPTGLTNEKLFFGSGTQLSVL

SEQ ID NO: 54 - protein, 107 aa, synthetic construct
  REGION 1..107: note = Synthetic construct
RKEVEQDPGPFNVPEGATVAFNCTYSNSASQSFFWYRQDCRKEPKLLMSVYSSGNEDGRFTAQLNRASQYISLLIRDSKLSDSATYLCVVRGQNFVFGPGTRLSVLP

SEQ ID NO: 55 - protein, 113 aa, synthetic construct
  REGION 1..113: note = Synthetic construct
NAGVTQTPKFQVLKTGQSMTLQCAQDMNHEYMSWYRQDPGMGLRLIHYSVGAGITDQGEVPNGYNVSRSTTEDFPLRLLSAAPSQTSVYFCASSYSDGNYGYTFGSGTRLTVV

SEQ ID NO: 56 - protein, 113 aa, synthetic construct
  REGION 1..113: note = Synthetic construct
EAQVTQNPRYLITVTGKKLTVTCSQNMNHEYMSWYRQDPGLGLRQIYYSMNVEVTDKGDVPEGYKVSRKEKRNFPLILESPSPNQTSLYFCASSTSDGNYGYTFGSGTRLTVV

SEQ ID NO: 57 - protein, 116 aa, synthetic construct
  REGION 1..116: note = Synthetic construct
AQKVTQAQTEISVVEKEDVTLDCVYETRDTTYYLFWYKQPPSGELVFLIRRNSFDEQNEISGRYSWNFQKSTSSFNFTITASQVVDSAVYFCALSYPGGGADGLTFGKGTHLIIQP

SEQ ID NO: 58 - protein, 115 aa, synthetic construct
  REGION 1..115: note = Synthetic construct
GAVVSQHPSRVICKSGTSVKIECRSLDFQATTMFWYRQFPKQSLMLMATSNEGSKATYEQGVEKDKFLINHASLTLSTLTVTSAHPEDSSFYICSAPDRVGYEQYFGPGTRLTVT

SEQ ID NO: 59 - protein, 111 aa, synthetic construct
  REGION 1..111: note = Synthetic construct
SQQGEEDPQALSIQEGENATMNCSYKTSINNLQWYRQNSGRGLVHLILIRSNEREKHSGRLRVTLDTSKKSSSLLITASRAADTASYFCATDSFGDYKLSFGAGTTVTVRA

SEQ ID NO: 60 - protein, 115 aa, synthetic construct
  REGION 1..115: note = Synthetic construct
DTGVSQDPRHKITKRGQNVTFRCDPISEHNRLYWYRQTLGQGPEFLTYFQNEAQLEKSRLLSDRFSAERPKGSFSTLEIQRTEQGDSAMYLCASSYDQGLAGIRYFGPGTRLTVT

SEQ ID NO: 61 - protein, 114 aa, synthetic construct
  REGION 1..114: note = Synthetic construct
AQSVTQLGSHVSVSEGALVLLRCNYSSSVPPYLFWYVQYPNQGLQLLLKYTSAATLVKGINGFEAEFKKSETSFHLTKPSAHMSDAAEYFCAVSASYNNNDMRFGAGTRLTVKP

SEQ ID NO: 62 - protein, 109 aa, synthetic construct
  REGION 1..109: note = Synthetic construct
TQLLEQSPQFLSIQEGENLTVYCNSSSVFSSLQWYRQEPGEGPVLLVTVVTGGEVKKLKRLTFQFGDARKDSSLHITAAQPGDTGLYLCALHGAQKLVFGQGTRLTINP

SEQ ID NO: 63 - protein, 113 aa, synthetic construct
  REGION 1..113: note = Synthetic construct
QKEVEQDPGPLSVPEGAIVSLNCTYSNSAFQYFMWYRQYSRKGPELLMYTYSSGNKEDGRFTAQVDKSSKYISLFIRDSQPSDSATYLCAMSDVWGGNEKLTFGTGTRLTIIP

SEQ ID NO: 64 - protein, 114 aa, synthetic construct
  REGION 1..114: note = Synthetic construct
ETGVTQTPRHLVMGMTNKKSLKCEQHLGHNAMYWYKQSAKKPLELMFVYSLEERVENNSVPSRFSPECPNSSHLFLHLHTLQPEDSALYLCASSPTDREYNEQFFGPGTRLTVL

SEQ ID NO: 65 - protein, 111 aa, synthetic construct
  REGION 1..111: note = Synthetic construct
KQEVTQIPAALSVPEGENLVLNCSFTDSAIYNLQWFRQDPGKGLTSLLLIQSSQREQTSGRLNASLDKSSGRSTLYIAASQPGDSATYLCAPQTSGSRLTFGEGTQLTVNP

SEQ ID NO: 66 - protein, 113 aa, synthetic construct
  REGION 1..113: note = Synthetic construct
GAGVSQSPSNKVTEKGKDVELRCDPISGHTALYWYRQSLGQGLEFLIYFQGNSAPDKSGLPSDRFSAERTGGSVSTLTIQRTQQEDSAVYLCASSLDRNYGYTFGSGTRLTVV

SEQ ID NO: 67 - protein, 109 aa, synthetic construct
  REGION 1..109: note = Synthetic construct
TQLLEQSPQFLSIQEGENLTVYCNSSSVFSSLQWYRQEPGEGPVLLVTVVTGGEVKKLKRLTFQFGDARKDSSLHITAAQPGDTGLYLCAGSDFNKFYFGSGTKLNVKP

SEQ ID NO: 68 - protein, 115 aa, synthetic construct
  REGION 1..115: note = Synthetic construct
KAGVTQTPRYLIKTRGQQVTLSCSPISGHRSVSWYQQTPGQGLQFLFEYFSETQRNKGNFPGRFSGRQFSNSRSEMNVSTLELGDSALYLCASSLMLAGSQETQYFGPGTRLLVL

SEQ ID NO: 69 - protein, 111 aa, synthetic construct
  REGION 1..111: note = Synthetic construct
DTAVSQTPKYLVTQMGNDKSIKCEQNLGHDTMYWYKQDSKKFLKIMFSYNNKELIINETVPNRFSPKSPDKAHLNLHINSLELGDSAVYFCASSLQGYEQYFGPGTRLTVT

SEQ ID NO: 70 - protein, 115 aa, synthetic construct
  REGION 1..115: note = Synthetic construct
DTAVSQTPKYLVTQMGNDKSIKCEQNLGHDTMYWYKQDSKKFLKIMFSYNNKELIINETVPNRFSPKSPDKAHLNLHINSLELGDSAVYFCASSQAGQGIFYGYTFGSGTRLTVV

SEQ ID NO: 71 - protein, 111 aa, synthetic construct
  REGION 1..111: note = Synthetic construct
GQSLEQPSEVTAVEGAIVQINCTYQTSGFYGLSWYQQHDGGAPTFLSYNALDGLEETGRFSSFLSRSDSYGYLLLQELQMKDSASYFCAVPFSGGYNKLIFGAGTRLAVHP

SEQ ID NO: 72 - protein, 117 aa, synthetic construct
  REGION 1..117: note = Synthetic construct
SAVISQKPSRDICQRGTSLTIQCQVDSQVTMMFWYRQQPGQSLTLIATANQGSEATYESGFVIDKFPISRPNLTFSTLTVSNMSPEDSSIYLCSVDLRDRGTTDTQYFGPGTRLTVL

SEQ ID NO: 73 - protein, 111 aa, synthetic construct
  REGION 1..111: note = Synthetic construct
KQEVTQIPAALSVPEGENLVLNCSFTDSAIYNLQWFRQDPGKGLTSLLLIQSSQREQTSGRLNASLDKSSGRSTLYIAASQPGDSATYLCAVNTGGFKTIFGAGTRLFVKA

SEQ ID NO: 74 - protein, 113 aa, synthetic construct
  REGION 1..113: note = Synthetic construct
DAGVIQSPRHEVTEMGQEVTLRCKPISGHDYLFWYRQTMMRGLELLIYFNNNVPIDDSGMPEDRFSAKMPNASFSTLKIQPSEPRDSAVYFCASSPGGGHLQYFGPGTRLTVT

SEQ ID NO: 75 - protein, 11 aa, synthetic construct
  REGION 1..11: note = Synthetic construct
ASSMLLNQPQH

SEQ ID NO: 76 - protein, 8 aa, synthetic construct
  REGION 1..8: note = Synthetic construct
QEAYKQQN

SEQ ID NO: 77 - protein, 6 aa, synthetic construct
  REGION 1..6: note = Synthetic construct
SQIVND

SEQ ID NO: 78 - protein, 7 aa, synthetic construct
  REGION 1..7: note = Synthetic construct
TSESDYY

SEQ ID NO: 79 - protein, 5 aa, synthetic construct
  REGION 1..5: note = Synthetic construct
LNHDA

SEQ ID NO: 80 - protein, 15 aa, synthetic construct
  REGION 1..15: note = Synthetic construct
AYNCPEPSDSSYKLI

SEQ ID NO: 81 - protein, 16 aa, synthetic construct
  REGION 1..16: note = Synthetic construct
VVSAVEDSGGGADGLT

SEQ ID NO: 82 - protein, 13 aa, synthetic construct
  REGION 1..13: note = Synthetic construct
ASSPILQGQYGYT

SEQ ID NO: 83 - protein, 7 aa, synthetic construct
  REGION 1..7: note = Synthetic construct
MTFSENT

SEQ ID NO: 84 - protein, 6 aa, synthetic construct
  REGION 1..6: note = Synthetic construct
VSPFSN

SEQ ID NO: 85 - protein, 11 aa, synthetic construct
  REGION 1..11: note = Synthetic construct
AYRSAGAQKLV

SEQ ID NO: 86 - protein, 14 aa, synthetic construct
  REGION 1..14: note = Synthetic construct
ASSLEGRDLIFEQF

SEQ ID NO: 87 - protein, 6 aa, synthetic construct
  REGION 1..6: note = Synthetic construct
YSYEKL

SEQ ID NO: 88 - protein, 5 aa, synthetic construct
  REGION 1..5: note = Synthetic construct
MGHRA

SEQ ID NO: 89 - protein, 15 aa, synthetic construct
  REGION 1..15: note = Synthetic construct
AGQRLETGGGADGLT

SEQ ID NO: 90 - protein, 12 aa, synthetic construct
  REGION 1..12: note = Synthetic construct
ASSSYRDNQPQH

SEQ ID NO: 91 - protein, 7 aa, synthetic construct
  REGION 1..7: note = Synthetic construct
LYKAGEL

SEQ ID NO: 92 - protein, 6 aa, synthetic construct
  REGION 1..6: note = Synthetic construct
SYDVKM

SEQ ID NO: 93 - protein, 5 aa, synthetic construct
  REGION 1..5: note = Synthetic construct
SIFNT

SEQ ID NO: 94 - protein, 5 aa, synthetic construct
  REGION 1..5: note = Synthetic construct
MDHEN

SEQ ID NO: 95 - protein, 12 aa, synthetic construct
  REGION 1..12: note = Synthetic construct
AVYPSGGYQKVT

SEQ ID NO: 96 - protein, 14 aa, synthetic construct
  REGION 1..14: note = Synthetic construct
ASSQDDSGAGNTIY

SEQ ID NO: 97 - protein, 6 aa, synthetic construct
  REGION 1..6: note = Synthetic construct
IYSNGD

SEQ ID NO: 98 - protein, 6 aa, synthetic construct
  REGION 1..6: note = Synthetic construct
DRGSQS

SEQ ID NO: 99 - protein, 11 aa, synthetic construct
  REGION 1..11: note = Synthetic construct
ASPGAGSYQLT

SEQ ID NO: 100 - protein, 13 aa, synthetic construct
  REGION 1..13: note = Synthetic construct
ASSPRVASVETQY

SEQ ID NO: 101 - protein, 7 aa, synthetic construct
  REGION 1..7: note = Synthetic construct
TSENNYY

SEQ ID NO: 102 - protein, 12 aa, synthetic construct
  REGION 1..12: note = Synthetic construct
ALSPEGFGNVLH

SEQ ID NO: 103 - protein, 14 aa, synthetic construct
  REGION 1..14: note = Synthetic construct
ASSQGASASYNEQF

SEQ ID NO: 104 - protein, 8 aa, synthetic construct
  REGION 1..8: note = Synthetic construct
RNSFDEQN

SEQ ID NO: 105 - protein, 6 aa, synthetic construct
  REGION 1..6: note = Synthetic construct
YNNKEL

SEQ ID NO: 106 - protein, 7 aa, synthetic construct
  REGION 1..7: note = Synthetic construct
TRDTTYY

SEQ ID NO: 107 - protein, 5 aa, synthetic construct
  REGION 1..5: note = Synthetic construct
LGHDT

SEQ ID NO: 108 - protein, 12 aa, synthetic construct
  REGION 1..12: note = Synthetic construct
AVHSGGGADGLT

SEQ ID NO: 109 - protein, 12 aa, synthetic construct
  REGION 1..12: note = Synthetic construct
ASSMMGQIDVLT

SEQ ID NO: 110 - protein, 5 aa, synthetic construct
  REGION 1..5: note = Synthetic construct
LSSGK

SEQ ID NO: 111 - protein, 5 aa, synthetic construct
  REGION 1..5: note = Synthetic construct
VGISA

SEQ ID NO: 112 - protein, 12 aa, synthetic construct
  REGION 1..12: note = Synthetic construct
AYRIMDSNYQLI

SEQ ID NO: 113 - protein, 13 aa, synthetic construct
  REGION 1..13: note = Synthetic construct
ASSTPRDLNTEAF

SEQ ID NO: 114 - protein, 6 aa, synthetic construct
  REGION 1..6: note = Synthetic construct
FNNNVP

SEQ ID NO: 115 - protein, 5 aa, synthetic construct
  REGION 1..5: note = Synthetic construct
SGHDY

SEQ ID NO: 116 - protein, 17 aa, synthetic construct
  REGION 1..17: note = Synthetic construct
AYRSGGGNYGGSQGNLI

SEQ ID NO: 117 - protein, 14 aa, synthetic construct
  REGION 1..14: note = Synthetic construct
ASSQEGLDTSYEQY

SEQ ID NO: 118 - protein, 10 aa, synthetic construct
  REGION 1..10: note = Synthetic construct
AVDTGGFKTI

SEQ ID NO: 119 - protein, 12 aa, synthetic construct
  REGION 1..12: note = Synthetic construct
ASSSGDGNYGYT

SEQ ID NO: 120 - protein, 7 aa, synthetic construct
  REGION 1..7: note = Synthetic construct
IQSSQRE

SEQ ID NO: 121 - protein, 6 aa, synthetic construct
  REGION 1..6: note = Synthetic construct
SMNVEV

SEQ ID NO: 122 - protein, 6 aa, synthetic construct
  REGION 1..6: note = Synthetic construct
DSAIYN

SEQ ID NO: 123 - protein, 5 aa, synthetic construct
  REGION 1..5: note = Synthetic construct
MNHEY

SEQ ID NO: 124 - protein, 11 aa, synthetic construct
  REGION 1..11: note = Synthetic construct
AVDSGATNKLI

SEQ ID NO: 125 - protein, 15 aa, synthetic construct
  REGION 1..15: note = Synthetic construct
SVDGGVGVTSTDTQY

SEQ ID NO: 126 - protein, 7 aa, synthetic construct
  REGION 1..7: note = Synthetic construct
ANQGSEA

SEQ ID NO: 127 - protein, 5 aa, synthetic construct
  REGION 1..5: note = Synthetic construct
SQVTM

SEQ ID NO: 128 - protein, 12 aa, synthetic construct
  REGION 1..12: note = Synthetic construct
ASEPPSNTGKLI

SEQ ID NO: 129 - protein, 14 aa, synthetic construct
  REGION 1..14: note = Synthetic construct
ASSQATLRGGDTQY

SEQ ID NO: 130 - protein, 12 aa, synthetic construct
  REGION 1..12: note = Synthetic construct
AVPFSGGYNKLI

SEQ ID NO: 131 - protein, 14 aa, synthetic construct
  REGION 1..14: note = Synthetic construct
SVDLRDRGTTDTQY

SEQ ID NO: 132 - protein, 6 aa, synthetic construct
  REGION 1..6: note = Synthetic construct
NALDGL

SEQ ID NO: 133 - protein, 6 aa, synthetic construct
  REGION 1..6: note = Synthetic construct
TSGFYG

SEQ ID NO: 134 - protein, 11 aa, synthetic construct
  REGION 1..11: note = Synthetic construct
ALAYSSASKII

SEQ ID NO: 135 - protein, 4 aa, synthetic construct
  REGION 1..4: note = Synthetic construct
HISR

SEQ ID NO: 136 - protein, 6 aa, synthetic construct
  REGION 1..6: note = Synthetic construct
YSGSPE

SEQ ID NO: 137 - protein, 13 aa, synthetic construct
  REGION 1..13: note = Synthetic construct
ASSPSLAGNEKLF

SEQ ID NO: 138 - protein, 11 aa, synthetic construct
  REGION 1..11: note = Synthetic construct
ASGTGDRETQY

SEQ ID NO: 139 - protein, 13 aa, synthetic construct
  REGION 1..13: note = Synthetic construct
ALTRATGGGNKLT

SEQ ID NO: 140 - protein, 11 aa, synthetic construct
  REGION 1..11: note = Synthetic construct
SAQDRIGYGYT

SEQ ID NO: 141 - protein, 7 aa, synthetic construct
  REGION 1..7: note = Synthetic construct
SNEGSKA

SEQ ID NO: 142 - protein, 6 aa, synthetic construct
  REGION 1..6: note = Synthetic construct
DFQATT

SEQ ID NO: 143 - protein, 14 aa, synthetic construct
  REGION 1..14: note = Synthetic construct
ALRKETGGGADGLT

SEQ ID NO: 144 - protein, 11 aa, synthetic construct
  REGION 1..11: note = Synthetic construct
SAQPREGYGYT

SEQ ID NO: 145 - protein, 9 aa, synthetic construct
  REGION 1..9: note = Synthetic construct
AFDNFNKFY

SEQ ID NO: 146 - protein, 13 aa, synthetic construct
  REGION 1..13: note = Synthetic construct
SARDGGQGTLEQY

SEQ ID NO: 147 - protein, 10 aa, synthetic construct
  REGION 1..10: note = Synthetic construct
AVDAGGFKTI

SEQ ID NO: 148 - protein, 12 aa, synthetic construct
  REGION 1..12: note = Synthetic construct
ASSLLDGNTEAF

SEQ ID NO: 149 - protein, 13 aa, synthetic construct
  REGION 1..13: note = Synthetic construct
ASSPVAKNSYEQY

SEQ ID NO: 150 - protein, 12 aa, synthetic construct
  REGION 1..12: note = Synthetic construct
AARTVSGTYKYI

SEQ ID NO: 151 - protein, 16 aa, synthetic construct
  REGION 1..16: note = Synthetic construct
ASSYSPSPGLASYEQY

SEQ ID NO: 152 - protein, 7 aa, synthetic construct
  REGION 1..7: note = Synthetic construct
IRPDVSE

SEQ ID NO: 153 - protein, 6 aa, synthetic construct
  REGION 1..6: note = Synthetic construct
SVGAGI

SEQ ID NO: 154 - protein, 6 aa, synthetic construct
  REGION 1..6: note = Synthetic construct
NTAFDY

SEQ ID NO: 155 - protein, 10 aa, synthetic construct
  REGION 1..10: note = Synthetic construct
AVNTGGFKTI

SEQ ID NO: 156 - protein, 13 aa, synthetic construct
  REGION 1..13: note = Synthetic construct
ASSTTGLANEKLF

SEQ ID NO: 157 - protein, 13 aa, synthetic construct
  REGION 1..13: note = Synthetic construct
ASAQDLSGANVLT

SEQ ID NO: 158 - protein, 13 aa, synthetic construct
  REGION 1..13: note = Synthetic construct
ALGGLTGGGNKLT

SEQ ID NO: 159 - protein, 10 aa, synthetic construct
  REGION 1..10: note = Synthetic construct
AADTGGFKTI

SEQ ID NO: 160 - protein, 12 aa, synthetic construct
  REGION 1..12: note = Synthetic construct
ASTGGDYNSPLH

SEQ ID NO: 161 - protein, 13 aa, synthetic construct
  REGION 1..13: note = Synthetic construct
ASSPTGLTNEKLF

SEQ ID NO: 162 - protein, 8 aa, synthetic construct
  REGION 1..8: note = Synthetic construct
VVRGQNFV

SEQ ID NO: 163 - protein, 10 aa, synthetic construct
  REGION 1..10: note = Synthetic construct
ASSLQGYEQY

SEQ ID NO: 164 - protein, 6 aa, synthetic construct
  REGION 1..6: note = Synthetic construct
VYSSGN

SEQ ID NO: 165 - protein, 6 aa, synthetic construct
  REGION 1..6: note = Synthetic construct
NSASQS

SEQ ID NO: 166 - protein, 12 aa, synthetic construct
  REGION 1..12: note = Synthetic construct
ASSYSDGNYGYT

SEQ ID NO: 167 - protein, 12 aa, synthetic construct
  REGION 1..12: note = Synthetic construct
ASSTSDGNYGYT

SEQ ID NO: 168 - protein, 13 aa, synthetic construct
  REGION 1..13: note = Synthetic construct
ALSYPGGGADGLT

SEQ ID NO: 169 - protein, 11 aa, synthetic construct
  REGION 1..11: note = Synthetic construct
SAPDRVGYEQY

SEQ ID NO: 170 - protein, 11 aa, synthetic construct
  REGION 1..11: note = Synthetic construct
ATDSFGDYKLS

SEQ ID NO: 171 - protein, 13 aa, synthetic construct
  REGION 1..13: note = Synthetic construct
ASSYDQGLAGIRY

SEQ ID NO: 172 - protein, 7 aa, synthetic construct
  REGION 1..7: note = Synthetic construct
IRSNERE

SEQ ID NO: 173 - protein, 6 aa, synthetic construct
  REGION 1..6: note = Synthetic construct
FQNEAQ

SEQ ID NO: 174 - protein, 5 aa, synthetic construct
  REGION 1..5: note = Synthetic construct
TSINN

SEQ ID NO: 175 - protein, 5 aa, synthetic construct
  REGION 1..5: note = Synthetic construct
SEHNR

SEQ ID NO: 176 - protein, 12 aa, synthetic construct
  REGION 1..12: note = Synthetic construct
AVSASYNNNDMR

SEQ ID NO: 177 - protein, 14 aa, synthetic construct
  REGION 1..14: note = Synthetic construct
ASSQAGQGIFYGYT

SEQ ID NO: 178 - protein, 8 aa, synthetic construct
  REGION 1..8: note = Synthetic construct
YTSAATLV

SEQ ID NO: 179 - protein, 6 aa, synthetic construct
  REGION 1..6: note = Synthetic construct
SSVPPY

SEQ ID NO: 180 - protein, 9 aa, synthetic construct
  REGION 1..9: note = Synthetic construct
ALHGAQKLV

SEQ ID NO: 181 - protein, 11 aa, synthetic construct
  REGION 1..11: note = Synthetic construct
ASSPGGGHLQY

SEQ ID NO: 182 - protein, 7 aa, synthetic construct
  REGION 1..7: note = Synthetic construct
VVTGGEV

SEQ ID NO: 183 - protein, 5 aa, synthetic construct
  REGION 1..5: note = Synthetic construct
SVFSS

SEQ ID NO: 184 - protein, 13 aa, synthetic construct
  REGION 1..13: note = Synthetic construct
AMSDVWGGNEKLT

SEQ ID NO: 185 - protein, 13 aa, synthetic construct
  REGION 1..13: note = Synthetic construct
ASSPTDREYNEQF

SEQ ID NO: 186 - protein, 6 aa, synthetic construct
  REGION 1..6: note = Synthetic construct
TYSSGN

SEQ ID NO: 187 - protein, 6 aa, synthetic construct
  REGION 1..6: note = Synthetic construct
YSLEER

SEQ ID NO: 188 - protein, 6 aa, synthetic construct
  REGION 1..6: note = Synthetic construct
NSAFQY

SEQ ID NO: 189 - protein, 5 aa, synthetic construct
  REGION 1..5: note = Synthetic construct
LGHNA

SEQ ID NO: 190 - protein, 10 aa, synthetic construct
  REGION 1..10: note = Synthetic construct
APQTSGSRLT

SEQ ID NO: 191 - protein, 11 aa, synthetic construct
  REGION 1..11: note = Synthetic construct
ASSLDRNYGYT

SEQ ID NO: 192 - protein, 6 aa, synthetic construct
  REGION 1..6: note = Synthetic construct
FQGNSA

SEQ ID NO: 193 - protein, 5 aa, synthetic construct
  REGION 1..5: note = Synthetic construct
SGHTA

SEQ ID NO: 194 - protein, 9 aa, synthetic construct
  REGION 1..9: note = Synthetic construct
AGSDFNKFY

SEQ ID NO: 195 - protein, 14 aa, synthetic construct
  REGION 1..14: note = Synthetic construct
ASSLMLAGSQETQY

SEQ ID NO: 196 - protein, 6 aa, synthetic construct
  REGION 1..6: note = Synthetic construct
YFSETQ

SEQ ID NO: 197 - protein, 5 aa, synthetic construct
  REGION 1..5: note = Synthetic construct
SGHRS